SEQUENCE LISTING (33 sequences)

SEQ ID NO: 1 - protein, 10 aa, Artificial Sequence
  synthetic
FFLLTRILTI

SEQ ID NO: 2 - protein, 15 aa, Artificial Sequence
  synthetic
FFLLTRILTIPQSLD

SEQ ID NO: 3 - protein, 15 aa, Artificial Sequence
  synthetic
TSLNFLGGTTVCLGQ

SEQ ID NO: 4 - protein, 16 aa, Artificial Sequence
  synthetic
QSPTSNHSPTSCPPIC

SEQ ID NO: 5 - protein, 14 aa, Artificial Sequence
  synthetic
CTTPAQGNSMFPSC

SEQ ID NO: 6 - protein, 8 aa, Artificial Sequence
  synthetic
WASVRFSW

SEQ ID NO: 7 - protein, 9 aa, Artificial Sequence
  synthetic
LLPIFFCLW

SEQ ID NO: 8 - protein, 9 aa, Artificial Sequence
  synthetic
VLQAGFFLL

SEQ ID NO: 9 - protein, 9 aa, Artificial Sequence
  synthetic
FLLTRILTI

SEQ ID NO: 10 - protein, 9 aa, Artificial Sequence
  synthetic
FLGGTPVCL

SEQ ID NO: 11 - protein, 9 aa, Artificial Sequence
  synthetic
LLCLIFLLV

SEQ ID NO: 12 - protein, 10 aa, Artificial Sequence
  synthetic
LVLLDYQGML

SEQ ID NO: 13 - protein, 10 aa, Artificial Sequence
  synthetic
LLDYQGMLPV

SEQ ID NO: 14 - protein, 9 aa, Artificial Sequence
  synthetic
WLSLLVPFV

SEQ ID NO: 15 - protein, 10 aa, Artificial Sequence
  synthetic
GLSPTVWLSV

SEQ ID NO: 16 - protein, 10 aa, Artificial Sequence
  synthetic
SIVSPFIPLL

SEQ ID NO: 17 - protein, 9 aa, Artificial Sequence
  synthetic
ILSPFLPLL

SEQ ID NO: 18 - protein, 10 aa, Artificial Sequence
  synthetic
LLVPFVQWFV

SEQ ID NO: 19 - protein, 10 aa, Artificial Sequence
  synthetic
FLPSDFFPSI

SEQ ID NO: 20 - protein, 10 aa, Artificial Sequence
  synthetic
FLPSDFFPSV

SEQ ID NO: 21 - protein, 9 aa, Artificial Sequence
  synthetic
CLTFGRETV

SEQ ID NO: 22 - protein, 9 aa, Artificial Sequence
  synthetic
EYLVSFGVW

SEQ ID NO: 23 - protein, 13 aa, Artificial Sequence
  synthetic
TPPATRPPNAPIL

SEQ ID NO: 24 - protein, 8 aa, Artificial Sequence
  synthetic
KYTSFPWL

SEQ ID NO: 25 - protein, 9 aa, Artificial Sequence
  synthetic
IPIPSSWAF

SEQ ID NO: 26 - protein, 11 aa, Artificial Sequence
  synthetic
WMMWYWGPSLY

SEQ ID NO: 27 - protein, 10 aa, Artificial Sequence
  synthetic
ILLLCLIFLL

SEQ ID NO: 28 - protein, 10 aa, Artificial Sequence
  synthetic
RWMCLRRFII

SEQ ID NO: 29 - protein, 11 aa, Artificial Sequence
  synthetic
RFSWLSLLVPF

SEQ ID NO: 30 - protein, 9 aa, Artificial Sequence
  synthetic
LYNILSPFL

SEQ ID NO: 31 - protein, 9 aa, Artificial Sequence
  synthetic
PFLPLLPIF

SEQ ID NO: 32 - protein, 9 aa, Artificial Sequence
  synthetic
PFVQWFVGL

SEQ ID NO: 33 - protein, 8 aa, Artificial Sequence
  synthetic
CTKPTDGN

(19) United States
(12) Patent Application Publication
(10) Pub. No.: US 2018/0246105 A1
(43) Pub. Date: Aug. 30, 2018

(54) IMMUNOLOGICAL TEST KIT FOR EVALUATING VACCINE EFFICACY AND STORAGE METHOD THEREOF

(71) Applicant: FUDAN UNIVERSITY, Shanghai (CN)

(72) Inventors: Bin WANG, Shanghai (CN); Chaofan LI, Shanghai (CN); Chenliang ZHOU, Shanghai (CN); Xian ZHOU, Shanghai (CN); Shuang WANG, Shanghai (CN)

(21) Appl. No.: 15/962,776

(22) Filed: Apr. 25, 2018

Related U.S. Application Data
(63) Continuation-in-part of application No. PCT/CN2016/101850, filed on Oct. 12, 2016.

(30) Foreign Application Priority Data
Oct. 26, 2015 (CN) 201510702998.4

(51) Int. Cl. G01N 33/576; G01N 33/68; G01N 33/53; G01N 33/49

(52) U.S. Cl. CPC G01N 33/5761; G01N 33/6857; G01N 33/5304; C12N 2730/10122; G01N 2333/70514; G01N 2333/70517; G01N 2333/70539; G01N 33/4915

(57) ABSTRACT

Embodiments of the invention provide a cell immunological assay kit used for evaluating the efficacy of a vaccine on the aspect of cell immunology and a method for storing the cell immunological assay kit. Some embodiments of the invention comprise a MHC-restricted viral antigenic peptide. Further, in some embodiments, the kit may use a vaccine research database of clinical trials and samples which are utilized in the therapeutic and conduct comprehensive testing on immune cells secrete cytokines by flow cytometry to establish a cellular immunology evaluation system. The kit may have a good storage stability and, in particular embodiments, the kit may provide a stability that maintains more than 90% of a material stored therein over a year or more.

### RELATED APPLICATIONS

This application is a continuation-in-part of co-pending PCT Application No. PCT/CN2016/101850 filed on Dec. 10, 2016 pursuant to 35 U.S.C. § 363, which claims priority from Chinese Application No. 201510702998.4 filed on Oct. 26, 2015, the entire contents of which are incorporated herein by reference.

### BACKGROUND

Currently the evaluation methods for prophylactic vaccines are relatively simple. Cohorts are generally used to evaluate antibody levels and population protection effectiveness, such as influenza vaccines, hepatitis B prophylactic vaccines, and smallpox vaccines. In the comprehensive evaluation of the effect of prophylactic vaccines, the antibody produced by the vaccine is often used as a core indicator. However, in the past decade with the strengthening of technology, especially the development of novel cell immunological assessment techniques, the evaluation of prophylactic vaccines also started to touch on important adaptive immunity cells, such as T cells. In the evaluation of prophylactic influenza, smallpox and other vaccines, except the detection of Immunoglobulin G (IgG), the cytokines such as IFN-γ and IL-2 expressed by T cells were detected, to determine the long-term effects and protective effects of the vaccine.

Therapeutic vaccines have been researched on many types of chronic infectious diseases, tumors, autoimmune diseases, and neurodegenerative diseases. In the past two to three decades of the development in the therapeutic vaccines, there have been many difficulties and, for at least this reason, only four therapeutic vaccines have been successfully marketed to date. One difficulty is not yet finding a universal immunological surrogate endpoint that may be used to predict the clinical efficacy of vaccines in the early stages of clinical research, such as the alternative endpoint of prophylactic vaccine evaluation-neutralizing antibodies. Due to the lack of this universal surrogate endpoint, the current evaluation of therapeutic vaccines has to rely on the final efficacy after clinical treatment, and the final clinical efficacy requires a large sample, and long-term, labor-intensive, cost-consuming IIb or III Phase clinical trials. Once the selected dose, usage, and/or strategy is insufficient, even if the therapeutic vaccine itself is indeed effective, it is unable to obtain a satisfied evaluation of efficacy, thus greatly delaying the listing of such products, and even killing some technologies and products.

Currently, the various key technologies of therapeutic vaccines have advanced largely. However, research on the parallel evaluation method for the surrogate endpoints related with final clinical outcomes is still nonexistent, with no unified, standardized, and/or comprehensive evaluation tools. Presently, clinical evaluation of therapeutic vaccines is relatively simple, mainly compromising clinical phenotypes changes, vaccines inducing antibodies, and immune memory capabilities. However, this simple evaluation may only give initial conclusions on the effectiveness of a therapeutic vaccine, and cannot indicate the overall impact of the immune system by the vaccine to predict the therapeutic effect, and cannot explain the root cause of the success and failure of the subject.

The overall impact on the immune system by a vaccine is mainly reflected in the impact on the various types of immune cells. However, evaluation of cell immunological indicators is still in an early stage with no unified standard. So, how to use biological markers on immune cells to evaluate therapeutic vaccines is a major issue for many current R&D fields.

In the exploration of the evaluation system, some studies focus on the human immunodeficiency virus (HIV) vaccine. With the therapeutic vaccine for HIV being continuously explored for more than 30 years, the evaluation system has made new progress. The initial evaluation indicators were HIV clinical phenotypes, including producing a wide range of HIV envelope specific neutralizing antibodies, viral load, and a number of CD4⁺ T cells changes. However, the therapeutic effect from the view of cell immunology cannot be predicted, the reasons for the effectiveness cannot be explained, and the direction of future improvement cannot be indicated. These problems greatly delay the listing of the vaccines and even kill effective vaccines. Presently, most of the HIV vaccines focus on the CD8⁺ T cells response and, therefore, functional cytokine, such as IFN-γ or TNF-α secreted by CD8⁺ T cells, is detected in the evaluation. However, this evaluation is not comprehensive, and the various indicators are independently detected to consume precious clinical samples.

With the further development of immunology and virology, it has been gradually recognized in the vaccines evaluation that a single group of lymphocytes or certain cytokines cannot completely evaluate the vaccine effect and explain the cause of success or failure. Therefore, for the clinical evaluation of HIV vaccine, during the treatment and the follow-up period, a researcher may comprehensively evaluate CD8⁺ T cell cytokines in the blood by a multi-color flow cytometry, in combination with related clinical phenotypes, and may identify one or more types of pluripotent CD8⁺ T cell subsets as a predictor for the evaluation. That has become a new development idea for the evaluation of HIV vaccines, and is being gradually applied in the evaluation of tuberculosis therapeutic vaccines, hepatitis B therapeutic vaccines, tumor therapeutic vaccines, and the like.

A Hepatitis B therapeutic vaccine is a developing area in therapeutic vaccine studies. The initial evaluation system mainly consists of HBV antigens, anti-HBV antibodies, HBV DNA levels and ALT levels, which only illustrate a protective or a therapeutic effect at the end of the clinical trial. But the efficacy may be impossible to predict and the reasons for success or failure may also be impossible. With more research being focused on vaccine evaluation, the levels of various cytokines in serum and the level of T cell responses may be researched. Studies have shown that HBV-specific CD8⁺ T cells can clear infected liver cells by secreting cytokines, mainly including interferon (IFN-γ), a tumor necrosis factor a (TNF-α) and direct cytotoxicity cytokines (perforin and granzymes) to induce cell death. Therefore, detection of CD8⁻ T cells function in vitro and evaluation of the level of cytokine expression may surmise the therapeutic effects to a certain extent. However, unlike HIV, the T cells of CHB patients may maintain virus-induced immune tolerance, and may be unable to express cytokines well in vitro after antigen re-stimulation. Therefore, how to activate CD8⁺ T cells to restore their ability in antigens response has become a difficult problem in evaluation in vitro. It has been found that the use of specific or non-specific enrichment methods in vitro may reactivate the activity of T cells well and may restore their ability.

Currently, more and more subgroups of T cells have been discovered, and the overall function of T cells may be the result of the interaction of various subgroups cells. One subgroup of T cells or one particular function subgroup may not fully reflect the overall impact of the immune system. With the further development of techniques, such as multicolor flow-type instruments, we may be able to detect ten or even more than twenty cytokines in one cell. Therefore, we cannot only detect the function of CTL cells, we may also detect the cytokine expression changes of Th cells and Treg cells after the vaccination, and comprehensively evaluate the vaccine for the overall impact of the expression of cytokines.

In the 1980s, Academician Wen Yumei of the Medical College of Fudan University led the lab to analyze the characteristics of HBV tolerance caused by mother-to-child transmission in most chronic hepatitis B patients in China. They considered that the patient's immune tolerance to hepatitis B surface antigen was the main mechanism for the chronicity of hepatitis B in China. According to the theory, they changed the way of antigen presentation though formation of new antigens to construct a hepatitis B therapeutic vaccine to eliminate the immune tolerance. In the study, the HBV surface antigen (HBsAg) plus human anti-HBs antibody vaccine-YIC (antigen-antibody-complex therapeutic Hepatitis B vaccine) were constructed. It was found that HBsAg was difficult to be recognized by dendritic cells (DC cells) in chronic hepatitis B patients, but a certain proportion of HBsAg-anti-HBs complex can mediate HBsAg into antigen-presenting cells via Fc receptors.

### SUMMARY OF THE INVENTION

Some embodiments of the invention provide a kit for evaluating a vaccine efficacy, and a method for storing the kit, and more particularly to a kit for evaluating a vaccine efficacy by a cell immunological assay and a method for storing the kit.

Some embodiments of the invention further provide an immunological evaluation of a vaccine efficacy in a new clinical stage IIIb. In particular, embodiments of the present invention provide a novel cell immunological assay kit for evaluating a vaccine efficacy. In one embodiment of the present invention, a cell immunological test kit for evaluating the efficacy of a vaccine is provided. In particular embodiments, the vaccine may comprise one or more MHC (Major Histocompatibility Complex) restriction viral antigen peptides. Further, the vaccine may be a therapeutic vaccine.

Among them, the virus may cause disease to humans and animals, and may be any one or more of a herpes virus, an influenza virus, a rabies virus, a variola virus, a hepatitis B virus, a hepatitis C virus, a hepatitis E virus, an HIV virus, and/or a human papillomavirus.

In a particular embodiment, the MHC-restricted viral antigen peptides may be CD4⁺ T cells hepatitis B surface antigen (HBsAg) epitope peptides and/or CD8⁺ T cell hepatitis B surface antigen (HBsAg) epitope peptides.

In a further embodiment, the CD4⁺ T cells HBsAg epitope peptides include, but are not limited to, any one or more of the following amino acid sequences: FFLLTRILTI (SEQ ID NO:1); FFLLTRILTIPQSLD (SEQ ID NO:2); TSLNFLGGTTVCLGQ (SEQ ID NO:3); QSPTSNHSPTSCPPIC (SEQ ID NO:4); CTTPAQGNSMFPSC (SEQ ID NO:5); CTKPTDGN (SEQ ID NO:33); WASVRFSW (SEQ ID NO:6); LLPIFFCLW (SEQ ID NO:7).

In a more particular embodiment, the CD8+ T cells HBsAg epitope peptide include, but are not limited to, any one or more of the following amino acid sequences: VLQAGFFLL (SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); PFVQWFVGL (SEQ ID NO:32).

In a further embodiment, the CD8⁺ T cell HBsAg epitope peptide may include, but is not limited to, any one or more of the following amino acid sequence groups A)-C): A) CD8⁺T cell A2 HBsAg epitope peptide VLQAGFFLL(SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17); B) CD8⁺T cell mixed HBV epitope peptide FLLTRILTI (SEQ ID NO:9); WLSLLVPFV (SEQ ID NO:14); GLSPTVWLSV (SEQ ID NO:15); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); C) CD8⁺ T cell non-HLA-A2-restricted HBsAg epitope peptides IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); PFVQWFVGL (SEQ ID NO:32).

In the embodiment above, the HBsAg epitope peptide for CD4⁺ T cells and the HBsAg epitope peptide for CD8⁺ T cells may be used alone or in combination. In the embodiment above, each group of the HBsAg epitope peptide for CD8⁺ T cells may be used alone or in combination of two or three groups. In one embodiment, the cell immunological test kit for evaluating the effect of a vaccine may further include measuring cell co-stimulatory signals.

In a particular embodiment, the co-stimulatory signals may be anti-CD3 and anti-CD28 antibodies. The cells to be tested according to embodiments of the present invention are preferably virus-specific T cells. The virus-specific T cells may be isolated from the virus-infected person. In the embodiments above, the subset of T cells may include helper T cells (e.g., Th1, Th2, and Th17), cytotoxic T cells (e.g., Tc1 and Tc17), and regulatory T cells (e.g., Treg and Tcreg).

In the embodiments above, the T cell marker molecules may include CD3, CD4, CD8, IFN-γ, TNF-α, IL-2, MIP-1β, IL-17A, IL-13, IL-10, IL-22, PD-1, Foxp3, TGF-β, IFN-α, IL-1β, IL-3, IL-4, IL-5, IL-6, IL-7, IL-8, IL-12p70, IL-15, IL-16, IL-21, IL-27, IL-29, IL-33, IP-10, MIP-1α, G-CSF, CXCL9, and the like.

The co-stimulatory signal may be any one or more commercially available anti-CD3 and anti-CD28 antibodies, such as Anti Human CD4 PerCp-Cy5.5, Anti Human CD8 PerCp-Cy5.5, Anti Human IFN-APC-Cy7, Anti Human TNF-APC, Anti Human IL-2 PE-CY7, Anti Human IL-17A FITC, Anti Human IL-13 FITC, Anti Human IL-10 PE-Cy7, Anti Human PD-1 APC-Cy7, one or more of Anti Human IL-22 PE, Anti Human Foxp3 APC, Anti Human MIP-1 FITC, Anti Human TGF-FITC.

In a particular embodiment, the cell immunological test kit for evaluating the effect of a vaccine may further include a protein transport blocker. In a further embodiment, the cell immunological test kit for evaluating the effect of the vaccine may further include any one or more of a pipetting device, one or more centrifuge tubes, and/or a cell culture vessel.

Some embodiments provide a cell culture container, such as 96 wells, pre-coating cell stimulation plate. In the embodiments herein, when the MHC-restricted epitope peptide are contacted with cells to be detected, the preferable concentration may be 1-20 μg/ml, more preferably may be 5-15μg/ml, and most preferably may be 8-10 μg/ml. In the embodiments above, when the anti-CD3 and anti-CD28 are contacted with the cells to be detected in the cell immunological assay, the preferable concentration may be 0.05-0.2μg/ml, more preferably may be 0.1-0.15μg/ml, such as 0.1 μg/ml and 0.05 μg/ml, 0.05 μg/ml and 0.05 μ/ml, 0.2 μg/ml and 0.05 μg/ml, 0.1 μg/ml and 0.1 μg/ml, 0.1 μg/ml and 0.2 m/ml, respectively.

According to another embodiment of the present invention, a method for storing a cell immunological test kit for evaluating the effect of a vaccine described above may be provided. In one embodiment of a storage method, the MHC-restricted viral antigen peptide storage temperature may be ≤5° C., more preferably may be ≤4° C., more preferably may be ≤3° C., and more preferably may be ≤0° C. It is preferably between 0° C. and 5° C. In the storage method, the cell culture vessel storage temperature may be <−10° C., more preferably may be ≤−15° C., more preferably may be ≤−20° C., and more preferably may be ≤−25° C. It is preferably between −30° C. and −10° C. or between −25° C. and −10° C. In the storage method, if other reagents and/or instruments are present, storage temperatures may be preferably ≤5° C., more preferably may be ≤4° C., more preferably may be ≤3° C., and more preferably may be ≤0° C. It is preferably between 0° C. and 4° C.

In one embodiment, the cell culture vessel storage temperature may be ≤−80° C. The Examples showed an example of the conditions and processes of cell cryopreservation at −80° C.

In some embodiments, the cell immunological test kit for evaluating the effect of a vaccine may utilize therapeutic vaccine research databases and/or specimens in a clinical trial stage, and may apply flow cytometry technology to comprehensively detect immune cells and their secreted cytokines, thereby establishing a cell immunological effect evaluation system. Further, in some embodiments, the cell immunological test kit for evaluating the effect of a vaccine may have a good stability and, in particular embodiments, the stability may maintain over 90% when stored for more than one year.

### DESCRIPTION OF THE DRAWINGS

FIG. 1 is an expression of IFN-γ in chronic hepatitis B patients after directly stimulated with the epitope peptides, without using the cell immunological test kit according to embodiments of the present invention. The positive stimulant is PMA+Iono, and the epitope peptides are the CD8 S epitope peptide pool, controls are unstimulated cells;

FIG. 2 is an expression of IFN-γ after cells of chronic hepatitis B patients were frozen, stimulated, and enriched for three days. The positive stimulant is PMA+Iono, and the stimulator of the experimental group is the CD8 S epitope peptide Pool, negative control is unstimulated cells;

FIG. 3 is an expression of IFN-γ after the PBMC of chronic hepatitis B patients were stimulated and enriched for three days, frozen, and resuscitation. The positive stimulation was PMA+Iono. The stimulus used in the experimental group was CD8 S epitope peptide. Negative controls were unstimulated cells after enrichment for three days;

FIG. 4A illustrates the changes in the proportion of different T cell subsets before and after treatment in Example 1, and shows a pie chart for the ratio of different CD4⁺ T cell subtypes during vaccination;

FIG. 4B illustrates the changes in the proportion of different T cell subsets before and after treatment in Example 1, and shows a pie chart for the ratio of different CD8⁺ T cell subsets during vaccination; the abscissa is the number of immunizations (0, 2, 4, and 6 immunizations); YIC: antigen-antibody-complex therapeutic Hepatitis B vaccine; ALUM: aluminum adjuvant group; SALINE: saline group;

FIG. 5A illustrates the changes of cytokines expression in different CD4⁺ T cells subgroups before and after treatment in Example 1, and shows the changes of CD4⁺ T cells cytokines during vaccination in the three treatment groups; YIC: antigen-antibody-complex therapeutic Hepatitis B vaccine, green; ALUM: aluminum adjuvant group, blue; SALINE: saline group, black;

FIG. 5B illustrates the changes of cytokines expression in different CD4⁺ T cells subgroups before and after treatment in Example 1, and shows the changes of IFN-γ produced by CD4⁺ T cells during vaccination. YIC group (left), aluminum adjuvant group (middle), and saline group (right); the abscissa was the number of immunizations (0, 2, 4 and 6 immunizations);

FIG. 6A shows the changes of cytokines in different subsets of CD8⁺ T cells before and after treatment in Example 1, and shows the trend of CD8⁺ T cells cytokines expression in the three treatment groups during vaccination; YIC: antigen-antibody-complex therapeutic Hepatitis B vaccine, green; ALUM: aluminum adjuvant group, blue; SALINE: saline group, black;

FIG. 6B shows the changes of cytokines in different subsets of CD8⁺ T cells before and after treatment in Example 1, and shows the secretion level of IL-2 in the CD8⁺ T cells during vaccination, YIC group (left), aluminum adjuvant group (middle), and saline group (right) and the abscissa was the number of immunizations (0, 2, 4 and 6 immunizations);

FIG. 7 illustrates a scheme of an immune therapy;

FIG. 8 illustrates a scheme of GM-CSF clinical trial completed;

FIG. 9 illustrates Serum HBsAg dynamics in patients with chronic hepatitis B. Zero weeks before immunotherapy was used as a baseline. Serum HBsAg was detected by ELISA. (A) HBsAg dynamics. Each dot may represent a patient. (B) In four groups each patient was treated with serum HBsAg changes. Color lines for each group of HBSAG transformation or sero-conversion of the HBsAg in patients;

FIG. 10 illustrates Serum ALT kinetics in patients with chronic hepatitis B. Zero weeks before immunotherapy being a baseline. Each dot may represent a patient;

FIG. 11 is a quality control chart of a clinical cellular immunology test. The PBMC were re-stimulated by PMA/Ionomycin (100 ng/mL/1 μg/mL), CMV (1 μg/mL) for 8 hours, and Golgi blocking BFA (1000×) was added in the last 4 hours. Intracellular staining was analyzed by flow cytometry;

FIG. 12 shows Foxp3 expression level of CD4⁺ T cells. The horizontal axis was 6 time points of treatment (0 weeks, 4 weeks, 12 weeks, 24 weeks, 36 weeks, 48 weeks) and 2 follow-up points (60 weeks, 72 weeks). The ordinate is CD4⁺Tcell Foxp3 expression level, each dot representing a single patient. Colors were labelled as ADV Group: Green; ADV+IFN-α group: purple; ADV+IFN-α+GM-CSF group: Blue; ADV+IFN-α+GM-CSF+vaccine Group: Orange. Statistics were shown by the mean±standard error (MEAN±SEM), *(p<0.05) indicates statistical significance, and ns (p>0.05) indicates no significance;

FIG. 13 illustrates IL-10 expression level of CD4⁺Tcells. The horizontal axis was 6 time points of treatment (0 weeks, 4 weeks, 12 weeks, 24 weeks, 36 weeks, 48 weeks) and 2 follow-up points (60 weeks, 72 weeks). The ordinate may be CD4⁺ T cell IL-10 expression level, each dot representing a single patient. Colors may be labelled as ADV Group: Green; ADV+IFN-α group: Purple; ADV+IFN-α+GM-CSF group: Blue; ADV+IFN-α+GM-CSF+vaccine Group: Orange. Statistics were shown by the mean±standard error (MEAN±SEM), * (p<0.05) indicates statistical significance, and ns (p>0.05) indicates no significance;

FIG. 14 illustrates IFN-γ, IL-2 and TNF-α of CD4⁺ T cells. The horizontal axis was 6 time points of treatment (0 weeks, 4 weeks, 12 weeks, 24 weeks, 36 weeks, 48 weeks) and 2 follow-up points (60 weeks, 72 weeks). The ordinate is CD4⁺T cell IFN-γ, IL-2, TNF-α expression levels, each dot representing a single patient. Colors were labelled as ADV Group: Green; ADV+IFN-α group: Purple; ADV+IFN-α+GM-CSF group: Blue; ADV+IFN-α+GM-CSF+vaccine Group: Orange. Statistics were shown by the mean±standard error (MEAN±SEM), *(p<0.05) indicates statistical significance, and ns (p>0.05) indicates no significance;

FIG. 15 shows IL-4 expression level of CD4⁺ T cells. The horizontal axis was 6 time points of treatment (0 weeks, 4 weeks, 12 weeks, 24 weeks, 36 weeks, 48 weeks) and 2 follow-up points (60 weeks, 72 weeks). The ordinate is CD4⁺ T cell IL-4 expression level, each dot representing a single patient. Colors were labelled as ADV Group: Green; ADV+IFN-α group: Purple; ADV+IFN-α+GM-CSF group: Blue; ADV+IFN-α+GM-CSF+vaccine Group: Orange. Statistics were shown by the mean±standard error (MEAN±SEM), *(p<0.05) indicates statistical significance, and ns (p>0.05) indicates no significance;

FIG. 16 illustrates IL-17 expression level of CD4⁺ T cells. The horizontal axis was 6 time points of treatment (0 weeks, 4 weeks, 12 weeks, 24 weeks, 36 weeks, 48 weeks) and 2 follow-up points (60 weeks, 72 weeks). The ordinate is CD4⁺ T cell IL-17 expression level, each dot representing a single patient. Colors were labelled as ADV Group: Green; ADV IFN-α group: Purple; ADV+IFN-α+GM-CSF group: Blue; ADV+IFN-α+GM-CSF+vaccine Group: Orange. Statistics were shown by the mean±standard error (MEAN±SEM), *(p<0.05) indicates statistical significance, and ns (p>0.05) indicates no significance;

FIG. 17 illustrates IFN-γ, IL-2, and TNF-α of CD8⁺ T cells. The horizontal axis was 6 time points of treatment (0 weeks, 4 weeks, 12 weeks, 24 weeks, 36 weeks, 48 weeks) and 2 follow-up points (60 weeks, 72 weeks). The ordinate is CD8⁺T cell IFN-γ, IL-2, TNF-α expression levels, each dot representing a single patient. Colors were labelled as ADV Group: Green; ADV+IFN-α group: Purple; ADV+IFN-α+GM-CSF group: Blue; ADV+IFN-α+GM-CSF+vaccine Group: Orange. Statistics were shown by the mean±standard error (MEAN±SEM), *(p<0.05) indicates statistical significance, and ns (p>0.05) indicates no significance;

FIG. 18 illustrates IL-17A of CD8⁺T cells. The horizontal axis was 6 time points of treatment (0 weeks, 4 weeks, 12 weeks, 24 weeks, 36 weeks, 48 weeks) and 2 follow-up points (60 weeks, 72 weeks). The ordinate is CD8⁺T cell IL-17A expression levels, each dot representing a single patient. Colors were labelled as ADV Group: Green; ADV+IFN-α group: Purple; ADV+IFN-α+GM-CSF group: Blue; ADV+IFN-α+GM-CSF+vaccine Group: Orange. Statistics were shown by the mean±standard error (MEAN±SEM), *(p<0.05) indicates statistical significance, and ns (p>0.05) indicates no significance;

FIG. 19 illustrates IFN-γ, IL-2, and TNF-α of CD8⁺T cells. The horizontal axis was 6 time points of treatment (0 weeks, 4 weeks, 12 weeks, 24 weeks, 36 weeks, 48 weeks) and 2 follow-up points (60 weeks, 72 weeks). The ordinate is CD8⁺T cell IFN-γ, IL-2, TNF-α expression levels, each dot representing a single patient. Colors were labelled as ADV Group: Green; ADV+IFN-α group: Purple; ADV+IFN-α+GM-CSF group: Blue; ADV+IFN-α+GM-CSF+vaccine Group: Orange. Statistics were shown by the mean±standard error (MEAN±SEM), *(p<0.05) indicates statistical significance, and ns (p>0.05) indicates no significance;

FIG. 20 illustrates IL-17A of CD8⁺T cells. The horizontal axis was 6 time points of treatment (0 weeks, 4 weeks, 12 weeks, 24 weeks, 36 weeks, 48 weeks) and 2 follow-up points (60 weeks, 72 weeks). The ordinate is CD8⁺T cell IL-17A expression levels, each dot representing a single patient. Colors were labelled as ADV Group: Green; ADV+IFN-α group: Purple; ADV+IFN-α+GM-CSF group: Blue; ADV+IFN-α+GM-CSF+vaccine Group: Orange. Statistics were shown by the mean±standard error (MEAN±SEM), *(p<0.05) indicates statistical significance, and ns (p>0.05) indicates no significance;

FIG. 21 illustrates the dynamics of HBsAg and Treg. The horizontal coordinates were 6 time points (0 weeks, 4 weeks, 12 weeks, 24 weeks, 36 weeks, 48 weeks) and 2 follow-up points (60 weeks, 72 weeks). The left ordinate was the HBsAg level, and the right ordinate was Treg level. Results were expressed by Mean±sem. Red line means HBsAg, black line indicates Treg;

FIG. 21 illustrates a correlation between HBsAg and Treg. The horizontal axis was Foxp3 of CD4+Tcells in 24 weeks. The ordinate axis was HBsAg level in 24 week. Correlation was calculated with Pearson analysis;

FIG. 22 illustrates the dynamics of Foxp3 and IFN-γ in CD4⁺ T cells. The horizontal axis was 6 time points of treatment (0 weeks, 4 weeks, 12 weeks, 24 weeks, 36 weeks, 48 weeks) and 2 follow-up points (60 weeks, 72 weeks). The left ordinate was the Foxp3 level in CD4⁺Tcells, and the right ordinate was the IFN-γ expression level in CD4⁺Tcells. Results were expressed in Mean±sem. Red lines represent IFN-γ⁺CD4⁺T cells, and black lines represent Foxp3⁺CD4⁺T cells;

FIG. 24 illustrates a correlation between Foxp3 and IFN-γ in CD4⁺T cells. The horizontal axis was Foxp3 of CD4⁺ T cells at 24 weeks. The ordinate axis was IFN-γ level in 24 week. Correlation was calculated with a Pearson analysis;

FIG. 25 illustrates the dynamics of Foxp3 in CD4⁺Tcells and IFN-γ in CD8⁺T cells. The horizontal axis was 6 time points of treatment (0 weeks, 4 weeks, 12 weeks, 24 weeks, 36 weeks, 48 weeks) and 2 follow-up points (60 weeks, 72 weeks). The left ordinate was the Foxp3 level in CD4⁺T cells, and the right ordinate was the IFN-γ expression level in CD8⁻T cells. Results were expressed in Mean±sem. Red lines represent IFN-γ⁺CD8⁺T cells, and black lines represent Foxp3⁺CD4⁺T cells;

FIG. 26 illustrates a correlation between Foxp3 in CD4⁺Tcells and IFN-γ in CD8⁺ T cells. The horizontal axis was Foxp3 of CD4⁺ T cells at 24 weeks. The ordinate axis was IFN-γ level in CD8⁺ T cells in 24 week. Correlation was calculated with a Pearson analysis;

FIG. 27 illustrates the dynamic change of the proportion of different T cell subsets. The horizontal axis is different time points (0 weeks, 4 weeks, 12 weeks, 24 weeks, 36 weeks, 48 weeks, 60 weeks, 72 weeks), and the ordinate axis is different treatment group;

FIG. 28 illustrates the dynamic change of TH1/Treg, Th2/Treg, Th17/Treg and Th1/Th2 ratios. The horizontal coordinates are different time points (0 weeks, 4 weeks, 12 weeks, 24 weeks, 36 weeks, 48 weeks, 60 weeks, 72 weeks), and the ordinate is TH1/Treg (A), Th2/Treg (B), Th17/Treg (C) and Th1/th2 (D) ratios. The statistic results are expressed in Mean±sem. ns (p>0.05) indicated no statistically significant differences, *(p<0.05) indicated statistically significant differences, and **(p<0.01) indicated statistically distinct differences;

FIG. 29A shows the stability of a cell immunoassay kit in the one-month, three-month, six-month, and 12-month storage in Example 2, and shows IFN-γ expression in CD8⁺ T cell after the kit stored at 1 month, 3 months, 6 months, and 12 months; Positive: Positive Stimulus (PMA+ionomycin); Negative: Negative Stimulus;

FIG. 29B shows the stability of a cell immunoassay kit in the one-month, three-month, six-month, and 12-month storage in Example 2, and shows the mean percentage of the stability of the cell immunoassay test kit stored at one month, three months, six months and 12 months, the abscissa is the preservation time; the number of samples is three chronic hepatitis B patients after the treatment of YIC.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The term “pharmaceutical composition,” as used herein, refers to any chemical or biological compound or substance, or a mixture or combination of two or more such compounds or substances, intended for use in the medical diagnosis, cure, treatment, or prevention of disease or pathology. In one embodiment, the present invention discloses a composition for evaluating vaccine efficacy comprising one or more MHC restrictive viral antigen peptides discussed herein and one or more biomarkers such as T cell makers for measuring cell co-stimulatory signals. In one embodiment, the MHC restrictive viral antigen peptides are CD4⁺ T cells HBsAg epitope peptides comprising or consisting of any one or more of the following amino acid sequences: FFLLTRILTI (SEQ ID NO:1); FFLLTRILTIPQSLD (SEQ ID NO:2); TSLNFLGGTTVCLGQ (SEQ ID NO:3); QSPTSNHSPTSCPPIC (SEQ ID NO:4); CTTPAQGNSMFPSC (SEQ ID NO:5); CTKPTDGN (SEQ ID NO:33); WASVRFSW (SEQ ID NO:6); and LLPIFFCLW (SEQ ID NO:7).

In a more particular embodiment, the MHC restrictive viral antigen peptides are the CD8+ T cells HBsAg epitope peptide comprising or consisting of any one or more of the following amino acid sequences: VLQAGFFLL (SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); PFVQWFVGL (SEQ ID NO:32).

In one embodiment, the MHC restrictive viral antigen peptides are combinations of one or more CD8+ T cells HBsAg epitope peptides disclosed herein with one or more CD4⁺ T cells HBsAg epitope peptides disclosed herein.

In one embodiments the biomarkers are T cell marker molecules such as CD3, CD4, CD8, IFN-γ, TNF-α, IL-2, MIP-1β, IL-17A, IL-13, IL-10, IL-22, PD-1, Foxp3, TGF-β, IFN-α, IL-1β, IL-3, IL-4, IL-5, IL-6, IL-7, IL-8, IL-12p70, IL-15, IL-16, IL-21, IL-27, IL-29, IL-33, IP-10, MIP-1α, G-CSF, CXCL9, and the like.

The term “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In one embodiment, the inventive compositions or methods can provide any amount of any level of treatment or prevention of a disease in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof. With respect to the inventive methods, the cancer can be any cancer, including any of the cancers associated with any of the tumor antigens described herein.

The present invention is applicable to any mammal. As used herein, the term “mammal” refers to a warm-blooded vertebrate animal such as a human, dog or cat or the like. In one embodiment, mammal includes rodents such as rodent, rabbit, feline, canine, swine, and cattle.

The terms “administer” or “administration,” as used herein, refers to their usual and ordinary meaning in the art of treating a patient with a substance such as a vaccine or a composition. The terms “co-administration” and “concomitant administration” as used herein are synonymous and refer to administering two substances or two compositions to a patient in such a manner and with such timing that both substances or both compositions reside in the patient's body at the same time. The co-administration may be simultaneous or sequential in time, and the co-administered substances or compositions may be administered to a patient at the same time, or separately but near in time, or on the same day, or otherwise in a way that results in substantial overlap of the residence periods for the respective substances or compositions in the body. The administration, e.g., parenteral administration, may include subcutaneous administration, intramuscular administration, transcutaneous administration, intradermal administration, intraperitoneal administration, intraocular administration, intranasal administration and intravenous administration.

The vaccine or the composition according to the invention may be administered to an individual according to methods known in the art. Such methods comprise application e.g. parenterally, such as through all routes of injection into or through the skin: e.g. intramuscular, intravenous, intraperitoneal, intradermal, mucosal, submucosal, or subcutaneous. Also, the vaccine may be applied by topical application as a drop, spray, gel or ointment to the mucosal epithelium of the eye, nose, mouth, anus, or vagina, or onto the epidermis of the outer skin at any part of the body. Other possible routes of application are by spray, aerosol, or powder application through inhalation via the respiratory tract. In this last case the particle size that is used will determine how deep the particles will penetrate into the respiratory tract. Alternatively, application may be via the alimentary route, by combining with the food, feed or drinking water e.g. as a powder, a liquid, or tablet, or by administration directly into the mouth as a: liquid, a gel, a tablet, or a capsule, or to the anus as a suppository.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and examples, and are not intended to limit the scope of embodiments of the invention. Skilled artisans will recognize the examples provided herein have many useful alternatives and fall within the scope of embodiments of the invention.

As described herein, a new hepatitis B therapeutic strategy was developed, which may include one dose of GM-CSF being injected every day for three consecutive days, and on the fourth day one dose of HBV vaccine may be injected in the same injection site (3×GM-CSF+VACCINE). The detailed pharmaceutical composition for viral immunotherapy and uses was described in U.S. application Ser. No. 15/007,160 (see also US 2016/0136265). This novel immunotherapy strategy may break the immune tolerance in HBV transgenic mice (Tg (Alb-1HBV) 44Bri/J). Both specific humoral and cellular immune were induced with the immunotherapy and 90% of the HBsAg was cleared in the transgenic mice. This immunotherapy strategy enter the clinical trial hosted by Zhejiang University, cooperation with Fudan University and other eight medical research institutions (No. 2013ZX10002001).

Currently, in addition to the clinical results, YIC and the new strategy of 3×GM-CSF+VACCINE are no specific immunological evaluation indicators or surrogate endpoints for the early and mid-term evaluation of the efficacy. Therefore, there may be a requirement for additional research on the immunological surrogate endpoint for chronic hepatitis B vaccine.

In one aspect, the present invention reveals a cellular immunology assay kit for evaluating the efficacy of vaccines against a virus.

In one embodiment, the cellular immunology assay kit comprises a MHC restrictive viral antigen peptide according to any embodiment as discussed herein. In one embodiment, the kit of the present invention may include a therapeutic device for delivering the MHC restrictive viral antigen peptide and/or one or more additional substance or compound. In one embodiment, the therapeutic device may be any suitable devices charged with a preparation of the MHC restrictive viral antigen peptide. In another embodiment, the therapeutic device may comprise any suitable devices charged with a preparation of the MHC restrictive viral antigen peptide and/or at least one additional substance or compound.

The instant invention may also include kits, packages and multicontainer units containing the above described pharmaceutical compositions, active ingredients, and/or means for administering the same for use in the evaluation of a vaccine efficacy against diseases and other conditions in mammalian subjects. Briefly, these kits include a container or formulation that contains the MHC restrictive viral antigen peptide, and/or other biologically active agents, possibly in combination with delivery enhancing agents disclosed herein formulated in a pharmaceutical preparation for delivery.

In one embodiment, the present invention provides a kit for evaluating a vaccine efficacy, a method for evaluating the vaccine efficacy, and a method for storing the kit, and more particularly to a kit for evaluating a vaccine efficacy by a cell immunological assay and a method for storing the kit.

Some embodiments of the invention further provide an immunological evaluation of a vaccine efficacy in a new clinical stage IIIb. In particular, embodiments of the present invention provide a novel cell immunological assay kit for evaluating a vaccine efficacy. In one embodiment of the present invention, a cell immunological test kit for evaluating the efficacy of a vaccine is provided. In particular embodiments, the vaccine may comprise one or more MHC (Major Histocompatibility Complex) restriction viral antigen peptides. Further, the vaccine may be a therapeutic vaccine.

Among them, the virus may cause disease to humans and animals, and may be any one or more of a herpes virus, an influenza virus, a rabies virus, a variola virus, a hepatitis B virus, a hepatitis C virus, a hepatitis E virus, an HIV virus, and/or a humans papillomavirus.

In a particular embodiment, the MHC-restricted viral antigen peptides may be CD4⁺ T cells hepatitis B surface antigen (HBsAg) epitope peptides and/or CD8⁺ T cell hepatitis B surface antigen (HBsAg) epitope peptides.

In a further embodiment, the CD4⁺ T cells HBsAg epitope peptides include, but are not limited to, any one or more of the following amino acid sequences: FFLLTRILTI (SEQ ID NO:1); FFLLTRILTIPQSLD (SEQ ID NO:2); TSLNFLGGTTVCLGQ (SEQ ID NO:3); QSPTSNHSPTSCPPIC (SEQ ID NO:4); CTTPAQGNSMFPSC (SEQ ID NO:5); CTKPTDGN (SEQ ID NO:33); WASVRFSW (SEQ ID NO:6); LLPIFFCLW (SEQ ID NO:7).

In one embodiment, the CD4⁺ T cells HBsAg epitope peptides comprise any one or more of the following amino acid sequences: FFLLTRILTI (SEQ ID NO:1); FFLLTRILTIPQSLD (SEQ ID NO:2); TSLNFLGGTTVCLGQ (SEQ ID NO:3); QSPTSNHSPTSCPPIC (SEQ ID NO:4); CTTPAQGNSMFPSC (SEQ ID NO:5); CTKPTDGN (SEQ ID NO:33); WASVRFSW (SEQ ID NO:6); or LLPIFFCLW (SEQ ID NO:7).

In one embodiment, the CD4⁺ T cells HBsAg epitope peptides consist essentially of any one or more of the following amino acid sequences: FFLLTRILTI (SEQ ID NO:1); FFLLTRILTIPQSLD (SEQ ID NO:2); TSLNFLGGTTVCLGQ (SEQ ID NO:3); QSPTSNHSPTSCPPIC (SEQ ID NO:4); CTTPAQGNSMFPSC (SEQ ID NO:5); CTKPTDGN (SEQ ID NO:33); WASVRFSW (SEQ ID NO:6); and LLPIFFCLW (SEQ ID NO:7).

In one embodiment, the CD4⁺ T cells HBsAg epitope peptides consist of any one or more of the following amino acid sequences: FFLLTRILTI (SEQ ID NO:1); FFLLTRILTIPQSLD (SEQ ID NO:2); TSLNFLGGTTVCLGQ (SEQ ID NO:3); QSPTSNHSPTSCPPIC (SEQ ID NO:4); CTTPAQGNSMFPSC (SEQ ID NO:5); CTKPTDGN (SEQ ID NO:33); WASVRFSW (SEQ ID NO:6); and LLPIFFCLW (SEQ ID NO:7).

In a more particular embodiment, the CD8+ T cells HBsAg epitope peptide include, but are not limited to, any one or more of the following amino acid sequences: VLQAGFFLL (SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); PFVQWFVGL (SEQ ID NO:32).

In one embodiment, the CD8+ T cells HBsAg epitope peptide comprise any one or more of the following amino acid sequences: VLQAGFFLL (SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); or PFVQWFVGL (SEQ ID NO:32).

In one embodiment, the CD8+ T cells HBsAg epitope peptide consist essentially of any one or more of the following amino acid sequences: VLQAGFFLL (SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); and PFVQWFVGL (SEQ ID NO:32).

In one embodiment, the CD8+ T cells HBsAg epitope peptide consist of any one or more of the following amino acid sequences: VLQAGFFLL (SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); and PFVQWFVGL (SEQ ID NO:32).

In a further embodiment, the CD8⁺ T cell HBsAg epitope peptide may include, but is not limited to, any one or more of the following amino acid sequence groups A)-C): A) CD8⁺ T cell A2 HBsAg epitope peptide VLQAGFFLL(SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17); B) CD8⁺ T cell mixed HBV epitope peptide FLLTRILTI (SEQ ID NO:9); WLSLLVPFV (SEQ ID NO:14); GLSPTVWLSV (SEQ ID NO:15); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); C) CD8⁺ T cell non-HLA-A2-restricted HBsAg epitope peptides IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); PFVQWFVGL (SEQ ID NO:32).

In the embodiment above, the HBsAg epitope peptide for CD4⁺ T cells and the HBsAg epitope peptide for CD8⁺ T cells may be used alone or in combination. In the embodiment above, each group of the HBsAg epitope peptide for CD8⁺ T cells may be used alone or in combination of two or three groups. In one embodiment, the cell immunological test kit for evaluating the effect of a vaccine may further include measuring cell co-stimulatory signals.

In a particular embodiment, the co-stimulatory signals may be anti-CD3 and anti-CD28 antibodies. The cells to be tested according to embodiments of the present invention are preferably virus-specific T cells. The virus-specific T cells may be isolated from the virus-infected person. In the embodiments above, the subset of T cells may include helper T cells (e.g., Th1, Th2, and Th17), cytotoxic T cells (e.g., Tc1 and Tc17), and regulatory T cells (e.g., Treg and Tcreg).

In the embodiments above, the T cell marker molecules may include CD3, CD4, CD8, IFN-γ, TNF-α, IL-2, MIP-1β, IL-17A, IL-13, IL-10, IL-22, PD-1, Foxp3, TGF-β, IFN-α, IL-1β, IL-3, IL-4, IL-5, IL-6, IL-7, IL-8, IL-12p70, IL-15, IL-16, IL-21, IL-27, IL-29, IL-33, IP-10, MIP-1α, G-CSF, CXCL9, and the like.

The co-stimulatory signal may be any one or more commercially available anti-CD3 and anti-CD28 antibodies, such as Anti Human CD4 PerCp-Cy5.5, Anti Human CD8 PerCp-Cy5.5, Anti Human IFN-APC-Cy7, Anti Human TNF-APC, Anti Human IL-2 PE-CY7, Anti Human IL-17A FITC, Anti Human IL-13 FITC, Anti Human IL-10 PE-Cy7, Anti Human PD-1 APC-Cy7, one or more of Anti Human IL-22 PE, Anti Human Foxp3 APC, Anti Human MIP-1 FITC, Anti Human TGF-FITC.

In a particular embodiment, the cell immunological test kit for evaluating the effect of a vaccine may further include a protein transport blocker. In a further embodiment, the cell immunological test kit for evaluating the effect of the vaccine may further include any one or more of a pipetting device, one or more centrifuge tubes, and/or a cell culture vessel.

Some embodiments provide a cell culture container, such as 96 wells, pre-coating cell stimulation plate. In the embodiments herein, when the MHC-restricted epitope peptide are contacted with cells to be detected, the preferable concentration may be 1-20 μg/ml, more preferably may be 5-15 μg/ml, and most preferably may be 8-10 μg/ml. In the embodiments above, when the anti-CD3 and anti-CD28 are contacted with the cells to be detected in the cell immunological assay, the preferable concentration may be 0.05-0.2 μg/ml, more preferably may be 0.1-0.15 μg/ml, such as 0.1 μg/ml and 0.05 μg/ml, 0.05 μg/ml and 0.05 μg/ml, 0.2 μg/ml and 0.05 μg/ml, 0.1 μg/ml and 0.1 μg/ml, 0.1 μg/ml and 0.2 μg/ml.

According to another embodiment of the present invention, a method for storing a cell immunological test kit for evaluating the effect of a vaccine described above may be provided. In one embodiment of a storage method, the MHC-restricted viral antigen peptide storage temperature may be ≤5° C., more preferably may be ≤4° C., more preferably may be ≤3° C., and more preferably may be ≤0° C. It is preferably between 0° C. and 5° C. In the storage method, the cell culture vessel storage temperature may be ≤−10° C., more preferably may be ≤−15° C., more preferably may be ≤−20° C., and more preferably may be ≤−25° C. It is preferably between −30° C. and −10° C. or between −25° C. and −10° C. In the storage method, if other reagents and/or instruments are present, storage temperatures may be preferably ≤5° C., more preferably may be ≤4° C., more preferably may be ≤3° C., and more preferably may be ≤0° C. It is preferably between 0° C. and 4° C.

In one embodiment, the cell culture vessel storage temperature may be ≤−80° C. The Examples showed an example of the conditions and processes of cell cryopreservation at −80° C. While it is generally required that liquid nitrogen is used to maintain temperature of −195.79 ° C. (Liquid nitrogen boiling point) or similar for cryopreservation, Applicants demonstrate that only −80 ° C., e.g., by using a refrigerator is sufficient to store cells and plasma in the present invention.

In some embodiments, the cell immunological test kit for evaluating the effect of a vaccine may utilize therapeutic vaccine research databases and/or specimens in a clinical trial stage, and may apply flow cytometry technology to comprehensively detect immune cells and their secreted cytokines, thereby establishing a cell immunological effect evaluation system. Further, in some embodiments, the cell immunological test kit for evaluating the effect of a vaccine may have a good stability and, in particular embodiments, the stability may maintain over 90% when stored for more than one year.

In one aspect, the present invention relates to a method for evaluating immunogenicity of a vaccine or immunotherapeutic composition by using the kit as discussed herein or the MHC restrictive viral antigen peptide composition as discussed herein.

In one embodiment, a method for evaluating immunogenicity of a vaccine or immunotherapeutic composition comprises the steps of: a) administering to a subject a vaccine or an immunotherapeutic composition; b) obtaining from the subject a sample; c) contacting the sample with a MHC restrictive viral antigen peptide composition; and d) evaluating immunogenicity of the subject.

Embodiments of the present invention will be described in detail with reference to figures and samples, but the implementation of the present invention is not limited thereto. Further, the experimental methods of the following examples herein are all conventional methods unless otherwise specified. The materials, reagents, and the like used in the following examples may be purchased from known biological companies, unless otherwise specified.

### Materials

(1) Therapeutic Hepatitis B Vaccine (commercial name: YIC) 60 μg/1 ml/ampoule, manufactured by Beijing Institute of Biological Products Co., Ltd., batch number: 20120301, 20100501.

(2) Aluminum hydroxide adjuvant injection: 1 ml 0.1% aluminum hydroxide adjuvant/ampule, appearance and general examination with the medication, provided by Beijing Institute of Biological Products Co., Ltd. Lot No.: 20120302, 20100801.

(3) Physiological saline injection: lml physiological saline/ampoule, provided by the Beijing Institute of Biological Products Co., Ltd. Lot number: 20110705.

(4) Adefovir dipivoxil tablets: The product with good efficacy in domestic market is provided by the sponsor. 100 mg/tablet, 14 tablets/box. Lot Nos.: 110980, 111195, 120661, 120871, 1211105, 130436, 1312102.

### EXAMPLES

### Example 1

### Cell Immunology Evaluation of Hepatitis B Therapeutic Vaccine (HBV Surface Antigen (HBsAg) Plus Human Anti-HBs Antibody:YIC)

Materials and Reagents

10 ml EDTA anticoagulation blood collection tube (BD, Cat. No.: 367525), cryotube (Corning, 430659), 15 ml centrifuge tube (Corning, 430791), 12-well cell culture plate (Costar, 3513), 96-well U bottom cell culture plate (Costar, 3799), 10 ml pipette (Costar, 4488), sterile 1.5 ml LEP tube, flow cytometer, and other consumables.

(1) A sterile phosphate buffer solution (PBS) was purchased from Gibco under the article number 20012-027. (2) Human lymphocyte separation solution (Lymphoprep™) was purchased from Axis-Shield Company, catalog number 11114547. (3) 4% paraformaldehyde (PFA) purchased from Sinopharm Chemical Reagents Co., Ltd. 8 g PFA was dissolved in PBS to a final volume of 200 mL, heated and stirred, and a few drops of concentrated NaOH was added, then cooled to room temperature, HCl was added to adjust the pH of the solution to 7.4 and stored at room temperature. (4) 0.2% cell membrane breaker (Triton X-100, purchased from Genview): 400 μl of TritonX-100 was added to PBS in a final volume of 200 ml, placed in a 60° C. water bath until completely dissolved (about 20 min.), cooled to room temperature, and saved at 4° C. (5) Double antibodies, fetal bovine serum (FBS) and RPMI 1640 medium were all purchased from Gibco Co., Ltd. with product numbers 10099-141 and 22400-089, respectively. (6) DMSO, PMA and Iono (Iono) were purchased from Sigma Corporation. (7) Protein transport blocker (BFA) was purchased from BD. (8) Anti-CD3 antibody (anti-CD3) and anti-CD28 antibody (anti-CD28) were purchased from Miltenyi Biotec. (9) Fluorescently labeled antibodies are shown in Table 1 below.

TABLE 1
Fluorescently labeled antibodies list

| The antibody name | Supplier |
|---|---|
| Anti Human CD4 PerCp-Cy5.5 | eBioscience |
| Anti Human CD8 PerCp-Cy5.5 | eBioscience |
| Anti Human IFN-γ APC-Cy7 | eBioscience |
| Anti Human TNF-γ APC | eBioscience |
| Anti Human IL-2 PE-CY7 | eBioscience |
| Anti Human IL-17A FITC | eBioscience |
| Anti Human IL-13 FITC | eBioscience |
| Anti Human IL-10 PE-Cy7 | eBioscience |
| Anti Human PD-1 APC-Cy7 | eBioscience |
| Anti Human IL-22 PE | eBioscience |
| Anti Human Foxp3 APC | eBioscience |
| Anti Human MIP-1β FITC | BD Biosciences |
| Anti Human TGF-β FITC | eBioscience |

The virus antigen peptides are shown in Tables 2, 3, and 4 below.

TABLE 2
CD4⁺ T cell HBsAg epitope peptide.

| Epitope | Sequence | HLA |
|---|---|---|
| S 19-28 | FFLLTRILTI (SEQ ID NO: 1) | DPw4/DR7 |
| S 19-33 | FFLLTRILTIPQSLD | DR2w 15 |
| | (SEQ ID NO: 2) | |
| S 37-51 | TSLNFLGGTTVCLGQ (SEQ ID | DR1 |
| | NO: 3) | |
| S 54-69 | QSPTSNHSPTSCPPIC (SEQ ID | |
| | NO: 4) | |
| S 124-137 | CTTPAQGNSMFPSC (SEQ ID | |
| | NO: 5) | |
| S 139-146 | CTKPTDGN (SEQ ID NO: 33) | DR1/3/4/5/6/ |
| | | 7/11 |
| S 165-172 | WASVRFSW (SEQ ID NO: 6) | DR11/14 |
| S 215-223 | LLPIFFCLW (SEQ ID NO: 7) | DR7/8/14 |

TABLE 3
CD8⁺ T cell HBV epitope peptide pool.

| Epitope | Sequence | HLA |
|---|---|---|
| S 14-22 | VLQAGFFLL (SEQ ID NO: 8) | A2 |
| S 20-28 | FLLTRILTI (SEQ ID NO: 9) | A2 |
| S 41-49 | FLGGTPVCL (SEQ ID NO: 10) | A2 |
| S 88-96 | LLCLIFLLV (SEQ ID NO: 11) | A2 |
| S 95-104 | LVLLDYQGML (SEQ ID | A2 |
| | NO: 12) | |
| S 97-106 | LLDYQGMLPV (SEQ ID | A2 |
| | NO: 13) | |
| S 172-180 | WLSLLVPFV (SEQ ID NO: 14) | A2 |
| S 185-194 | GLSPTVWLSV (SEQ ID | A2 |
| | NO: 15) | |
| S 207-216 | SIVSPFIPLL (SEQ ID NO: 16) | A2 |
| S 208-216 | ILSPFLPLL (SEQ ID NO: 17) | A2 |

TABLE 4
CD8⁺ T cell non-HLA-A2-restricted HBsAg
epitope peptide

| Sequence | HLA |
|---|---|
| IPIPSSWAF (SEQ ID NO: 25) | A33, B7 |
| WMMWWGPSLY (SEQ ID NO: 26) | A68 |
| ILLLCLIFLL (SEQ ID NO: 27) | A1, A29 |
| RWMCLRRFII (SEQ ID NO: 28) | A23, A24 |
| RFSWLSLLVPF (SEQ ID NO: 29) | A23, A24 |
| LYNILSPFL (SEQ ID NO: 30) | A24 |
| PFLPLLPIF (SEQ ID NO: 31) | A24 |
| PFVQWFVGL (SEQ ID NO: 32) | A24 |

Experimental Method

Research design and object: The study used random, multicenter, concomitant medications. The subjects were HBeAg-positive patients with chronic viral hepatitis B. The total number of planned enrollment cases was 60. The subjects were randomly assigned to the following three groups according to a ratio of 1:1:1.

TABLE 5
The grouping and treatment strategies

| | | |
|---|---|---|
| Adjuvant | 0.1% aluminum hydroxide adjuvant + | withdrawal |
| aluminum | adefovir dipivoxil for 24 weeks | follow-up 24 |
| group | | weeks |
| YIC group | 60 μg of YIC group + adefovir | withdrawal |
| | dipivoxil for 24 weeks | follow-up 24 |
| | | weeks |
| Saline group | saline + adefovir dipivoxil for 24 weeks | withdrawal |
| | | follow-up by 24 |
| | | weeks |

At the end of the study, 52 patients were enrolled in the study, including 18 patients in the aluminum adjuvant group, 17 patients in the YIC group, and 17 patients in the saline group. The subjects were intramuscularly injected with YIC, aluminum adjuvant, or saline respectively, every four weeks, totalling six times. The treatment was 24 weeks followed by a follow-up of 24 weeks. The overall study time was 48 weeks. For ethical considerations, all subjects were treated with the antiviral drug adefovir dipivoxil. Blood was collected at 0, 4, 12 and 20 weeks.

Blood collection, peripheral blood mononuclear cell (PBMC) isolation and T cells enrichment in vitro: (1) Collect 10 ml of whole blood with BD EDTA anticoagulant tube. After taking blood, invert and mix 5-10 times, place it standing upright, store it at room temperature for transportation, and process the test within 24 hours. (2) The blood collection tube is placed in a centrifuge and centrifuged at room temperature (1600 rpm) for 10 min. The upper layer of plasma is taken and packed at 1 ml/tube and stored at −70° C. until serological detection. (3) Add 5 ml preheated lymphocyte separation solution in two 15 ml centrifuge tubes respectively. (4) Mix the remaining blood after centrifugation in step 2 with sterile PBS to a final volume of 20 ml, and add 10 ml along the wall of the tube to the surface of 5 ml of lymphocyte separation solution to avoid blood rushing to the bottom of the tube. (5) Place the centrifuge tube in the horizontal rotor and adjust the centrifuge speed to the lowest (climb speed 0), and centrifuge at 22-23° C. (rpm 2000 rpm) for 30min. (6) Pipette the leukocyte layer cells (this is PBMC), and place the extracted liquids in two new 15 ml centrifuge tubes, add sterile PBS to a final volume of 15 ml, and resuspend them. Centrifuge at a horizontal rotation of 22-23 ° C. (2000 rpm) 5min, abandoned the supernatant. This wash step can be repeated once. (7) The washing step 6 can be repeated once. (8) After resuspension of the cells with 3 mL of complete medium R10 (RPMI 1640 medium containing 10% FBS, 100 U/ml penicillin, 100 μg/ml streptomycin), the cells were counted. (9) Adjust cell suspension concentration to 5×10⁶ cells/ml with R10 medium. (10) Take 3 ml of the cells from step 9, add anti-CD3 (final concentration 0.14ml) and anti-CD28 (final concentration 0.05 μg/ml), and then place 1 ml/well into a 12-well cell culture plate. Incubate in a cell incubator for 3 days.

However, based on the disclosure of the embodiments of the present invention, the technicians in this field can determine other anti-CD3 and anti-CD28 antibody stimulation concentrations, such as 0.05 μg/mL and 0.05 μg/mL, 0.2 μg/mL and 0.05 μg/mL, 0.1 μg/mL and 0.1 μg/mL and 0.1₁.tg/mL and 0.2 μg/mL, etc.

However, based on the disclosure content of embodiments of the present invention, the technicians in this field can determine other enrichment time, such as one day, five days, and seven days. However, based on the disclosure of embodiments of the present invention, the technicians in this field can determine that available subpopulations include helper T cells (Th1, Th2 and Th17), killer T cells (Tc1 and Tc17), and regulatory T cells (Treg and Tcreg).

In vitro specific stimulation of antigenic peptides: (1) After 3 days, the enriched cells were washed once with R10 medium, resuspended. 100₁11/well of anti-CD28 (final concentration 0.1 μg/ml) and BFA (1₁11/m1) were added. (2) Positive control group, antigen peptide stimulation group and blank control group were set in 96-well pre-coated cell culture plates. The positive control group was added with PMA (final concentration 0.1 μg/ml) and ionomycin (final concentration 1 μg/m1); the antigen peptide stimulation group was added with CD4/CD8 peptide pool for HBsAg (the final concentration of each peptide was 10 m/ml, respectively). However, the technicians in this field may determine the stimulatory concentrations of other antigen peptides on the basis of the disclosure of embodiments of the present invention, such as 1 μg/ml, 5 μg/ml and 20 μg/ml. (3) Incubate at 37° C. in a 5% CO₂ cell incubator for 8 hours. However, based on the disclosure of embodiments of the present invention, the technicians in this field can determine other stimulation times, such as 4 hours, 6 hours, and 12 hours.

Cell cryopreservation: (1) Centrifuge the whole 96-well plate (2000 rpm) at 22-23° C. for 5min and discard the supernatant. (2) Add 200 μl/well of cryopreservation solution (10% DMSO+90% fetal bovine serum), resuspend, and then cool by gradient: 4° C. for 1 hour, −20° C. for 40 minutes, and freeze at −80° C. To be unified batch detection.

Flow detection: (1) Rapidly thaw the cells in 96-well cell culture plates at 37° C. After centrifugation at 1500 rpm for 5 min, the supernatant was discarded, and 200 μl of PBS (surface dilution) added with 2% FBS was used to wash once, centrifuged at 1500 rpm for 5 min, and the supernatant was discarded. (2) The surface staining antibody was diluted with surface diluent, 50 μl per well, blotted and mixed 3-5 times, and protected from light for 30 min on ice. (3) Add 150 μl of the surface diluent to stop surface staining, centrifuge at 1500 rpm for 5 min, and discard the supernatant. (4) Add 200 μl 1 4% PFA for 8 min at room temperature to avoid light, centrifuge at 2000 rpm for 5 min, discard the supernatant, quickly add 200 μl of surface diluent to resuspend the cells, centrifuge at 2000 rpm for 5 min, and discard the supernatant. (5) Pre-assembled with 0.2% Triton X-100 (including 2% FBS) as a diluent for intracellular staining antibodies, 50 μl per well, pipetted and mixed, protected from light and stained on ice for 2 hours. (6) Stop the reaction with 150 μl well of surface dilution, centrifuge at 2000 rpm for 5 min, and discard the supernatant. (7) Resuspend the cells with 200 μl/well of surface dilution and transfer to a flow tube for detection. (8) Detection indicators: T cell surface markers: anti-CD4, anti-CD8, but the technicians in this field can determine other T cell surface markers such as anti-CD3 based on the disclosure of the present invention. Functionally related cell markers: anti-IFN-γ, anti-TNF-α, anti-IL-2, anti-MIP-1β, anti-IL-17A, anti-IL-13, anti-IL-10, anti-IL-22, anti-PD-1, anti-Foxp3 and anti-TGF-β, but the technicians in this field can determine other functional related cell markers based on the disclosure of the present invention, such as anti-IFN-α, anti-IL-1β, anti-IL-3, anti-IL-4, anti-IL-5, anti-IL-6, anti-IL-7, anti-IL-8, anti-IL-12p70 , anti-IL-15, anti-IL-16, anti-IL-21, anti-IL-27, anti-IL-29, anti-IL-33, anti-IP-10, anti-MIP-1α, anti -G-CSF and anti-CXCL9. (9) The cells were detected by the LSR Fortessa multicolor flow cytometer (BD Biosciences, USA) and were first compensated with a single fluorescent stain. The analysis was performed using FlowJo 7.6.1 (TreeStar, US) and the results showed the percentage of positive cells.

Statistical Analysis: T-tests were used for data analysis between the two groups. One-way ANOVA was used to detect more than two groups of data. Differences of P<0.05 were considered statistically significant. *indicates P<0.05.

Experimental Results

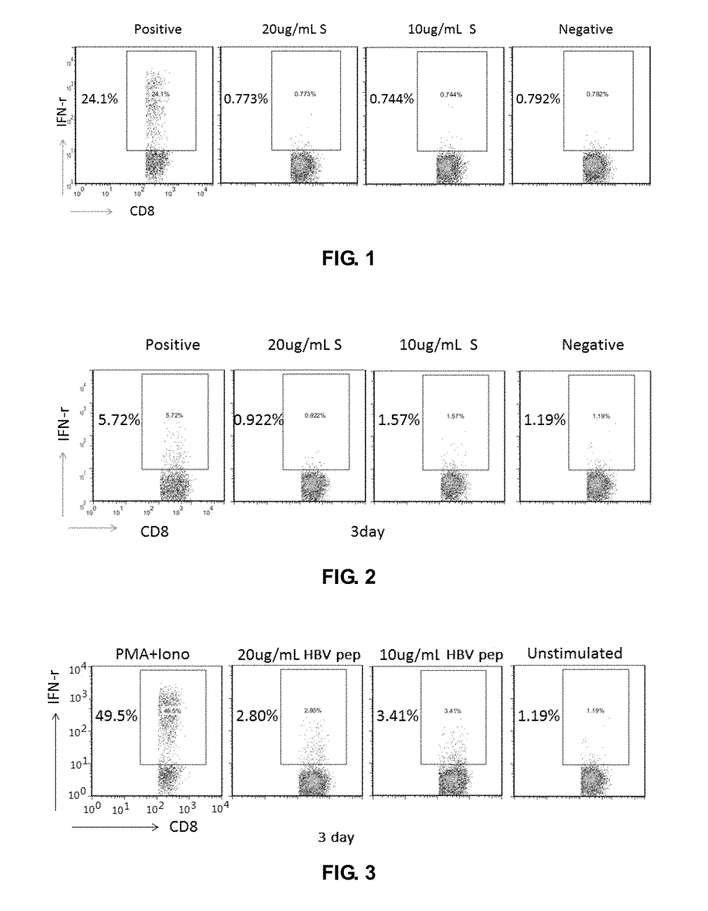

Because of the T cells tolerance to HBV antigens that is observed in patients with chronic hepatitis B infection, in order to explore suitable conditions for T cells detection, the PBMCs are pretreated. We have explored the stimulation and cryopreservation protocols, using the IFN-γ expression of CD8⁺ T as an example. We stimulated PBMCs of chronic hepatitis B patients by 10 μg/mL and 20 μg/mL S antigen epitope peptide without enrichment and cryopreservation. FIG. 1 showed that PBMCs without any pretreatment did not respond positively to S-epitope peptides. After finding the suitable enrichment conditions, we selected the cells were frozen first or enriched first. It was found that if the PBMCs of the patients were frozen before enrichment and stimulation, IFN-γ expression of CD8⁺ T cells was decreased as a whole, and there was no obvious positive reaction (FIG. 2); if the PBMCs were enriched and stimulated before cryopreservation, IFN-γ expression of CD8⁺ T in patients with chronic hepatitis B after the cells recovery was significantly increased compared with the unstimulated group after stimulation with S epitope peptides (FIG. 3). It was proved that the method provided in the kit instructions can effectively detect S antigen specific-T cells response in chronic hepatitis B patients.

In order to evaluate the vaccine efficacy, we studied the changes of the proportion of different T cell subtypes during vaccination with YIC, aluminum adjuvant, and saline respectively, and classified T cells cytokines according to different immune functions in the immune response. CD4⁺ T cells cytokines are classified into Th1, Th2, Th17, and Tregs. Thl cytokines play an important role in antiviral activity, Th2 cytokines play a major role in humoral immunity, Th17 cytokines are mainly the inflammatory factors, and Treg cytokines mainly play an immunosuppressive role. The CD8⁻ T cell factors are classified into Tc1, Tc17, and Tcreg. Tc1 cytokines mainly act as killers, Tc17 is mainly inflammatory cytokines, and Tcreg mainly exerts immunosuppressive effects.

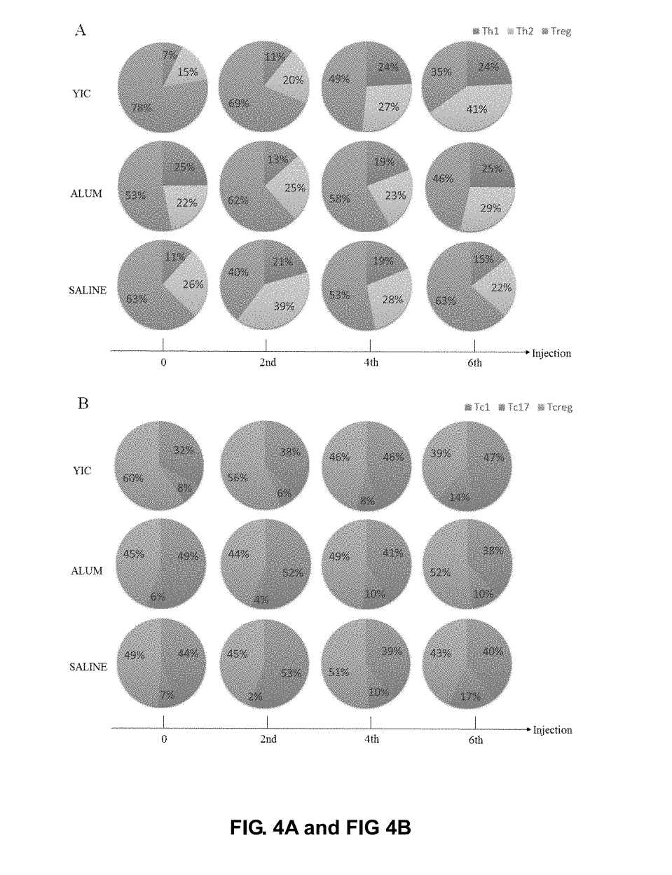

As shown in FIG. 4A, for CD4⁻ T cells, the proportion of Treg cells in YIC group was reduced from 78% at baseline to 35% at the end of treatment, and the proportion of Th1 cells was increased from 7% to 24%. The proportion of Th2 cells was increased from 15% to 41%; similar changes were not observed in aluminum adjuvant and saline group, and the proportion of Th1, Th2, and Treg cells did not change substantially during vaccination. As shown in FIG. 4B, for CD8⁺ T cells, the proportion of Tc1 and Tc17 cells in YIC group were increased, and the Tcreg ratio was decreased from 60% at baseline to 39% at the end of treatment. The proportion of Tc17 cells in aluminum group was increased slightly (from 8% at baseline to 14% at the end of the treatment), and the proportion of Tc17 cells in the saline group showed irregularities during vaccination. Changes of the ratio of Tc1 cells in the aluminum-adjuvant and saline-treated groups were all decreased, and the proportion of Tcreg cells was increased.

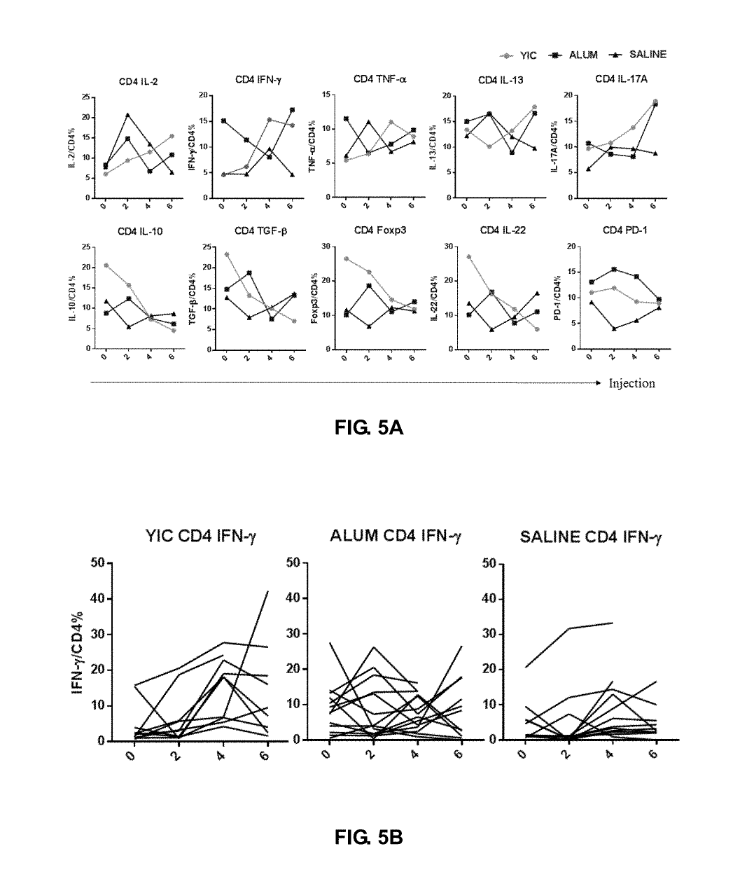

The expression changes of Th1 cells cytokines was further analyzed: IL-2, IFN-γ and TNF-β, Th2 cells: IL-13, Th17 cells: IL-17A , Treg cell: IL-10, TGF-β, Foxp3 and inhibitory molecules IL-22 and PD-1 after 0, 2, 4 and 6 immunizations in YIC group, the aluminum adjuvant group and the saline group. Based on the average expression levels of cytokines in the three treatment groups at different times of immunization, the result showed the changes in the expression levels of various characteristic markers during vaccination (FIG. 5A). The levels of IL-2, IFN-γ, and TNF-α in CD4⁺ T cells in the YIC group were increased, while those in the aluminum adjuvant group and the saline group showed irregular changes. The expression level of the five inhibitory factors in CD4⁺ T cells in YIC group had a decreasing trend, while those in the aluminum adjuvant group increased first and then decreased, showed irregular changes.

At the same time, according to the level of expression of CD4⁺ T cell IFN-γ at different times of immunization for each subject in YIC group, the aluminum adjuvant group, the saline group, and each of the three treatment groups were analyzed. The level of secretion of cytokines varies with vaccination (FIG. 5B).

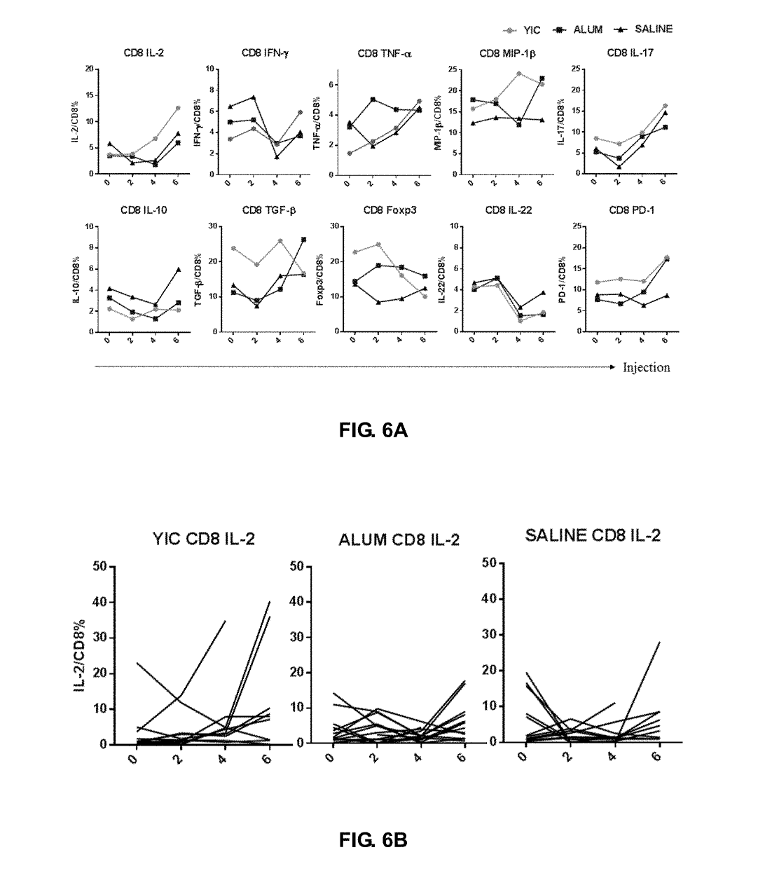

The changes of cytokines in CD8⁺T cells was studied and the cytokines IL-2, IFN-γ, TNF-α and MIP-1β in Tc1 cells, IL-17A, IL-10, TGF-β and transcriptional regulatory factor Foxp3 in Treg, and inhibitory molecules IL-22 and PD-1 at 0, 2, 4, 6 after 0, 2, 4 and 6 immunizations in YIC group, the aluminum adjuvant group, and the normal saline group were analyzed. According to the average of the expression levels of these cytokines in the three treatment groups at different times of immunization, FIG. 6A showed the changes in the expression levels of various characteristic markers. The expression of IL-2, IFN-γ, and TNF-α in CD8⁺ T cells in YIC group was increased. The expression levels of such in the aluminum adjuvant group and the saline group were irregular changes. The level of TGF-β, Foxp3, and IL-22 expression in CD8⁺ T cells in YIC group decreased. The expression of PD-1 was increased and the expression level of IL-10 did not change. TGF-β, PD-1 expression levels in the aluminum adjuvant group increased significantly, IL-10, Foxp3 and IL-22 expression levels did not change significantly; saline five groups. The expression of inhibitory factors did not change substantially.

At the same time, according to the level of expression of IL-2 in CD8⁺ T cells at different times of immunization for each subject in YIC group, the aluminum adjuvant group, the saline group, and each of the three treatment groups were analyzed. The level of secretion of these factors varies with the immune process, as shown in FIG. 6B.

### Example 2

### The Cell Immunology Evaluation for Hepatitis B Therapeutic Vaccine (ADV+IFN-α+GM-CSF+Vaccine)

1. Materials and Methods

1.1 Clinical Experiment Design and Enrollment of CHB Patients

Based on the principle of GCP, the clinical experiment was designed as multi-centered, randomized, and controlled. The drugs used in the experiment include Nucleoside Analogues (NA), Adefovir Dipivoxil (ADV), Interferon-α-2b (IFN-α), Granulocyte-Macrophage Colony-Stimulating Factor (GM-CSF) and HBV vaccine (VACCINE). The clinical experiment was registered in Chinese Clinical Trial Registry (Registered code: ChiCTR-TRC-13003254; Registered name: Increase HBsAg Clearance Rate in HBeAg Positive Chronic Hepatitis B Patients with New Antiviral/Immunomoduratory Therapy; Website: http://www.chictr.org. cn/showproj.aspx?proj=6305). The clinical experiment was supervised by the ethics committee of Huashan Hospital, Fudan University. All enrolled patients were signed in with an informed consent form.

The experiment was conducted in Huashan Hospital and 104 CHB patients were enrolled. The patients already received clinical treatment before enrollment. Patients were randomly divided into 4 groups and the treatment lasted for 48 weeks. Patients were seen for a follow-up visit twice on weeks 60 and 72. All the patients must be treated with NA for at least 71 weeks.

Names of the divided group: (1) Keep treatment with NAa; (2) ADV +IFN-α; (3) ADV+IFN-α+GM-CSF; (4) ADV+IFN-α+GM-CSF+VACCINE.

a: This group will named ADV below.

1.2 Inclusion and Exclusion Criteria

Inclusion criteria: Aged 18 to 65 years old, males or females; CHB patients whose HBeAg serotype transformed to negative, HBV-DNA cannot be detected, ALT returned to normal level after treatment with NA. The patients must also be given consolidation therapy for at least one year. The NA used for the treatment can include, singly or arbitrary combination, Lamivudine, ADV and Entecavir.

Exclusion criteria: Patients who were infected with Hepatitis A virus, Hepatitis C virus, Hepatitis D virus, Hepatitis E virus and HIV; Patients who have an allergic reaction to NA, Interferon, GM-CSF and HBV Vaccine; Patients with cirrhosis or Alpha fetoprotein level over 100 ng/ml; Patients with other liver related disease which include autoimmune liver disease, alcoholic liver disease, non-alcoholic fatty liver disease, drug-induced hepatitis and so on; Pregnant or lactating women; Patients whose serum creatinine was above the normal level, hemoglobin under 100 g/L, platelet count under 80×10⁹/L, neutrophil count under 1.5×10⁹/L; Patients with diseases in heart, brain, kidney, retina and muscle; Patients with endocrine system disease, autoimmune disease and malignancy.

1.3 Random Grouping Plan

Qualified experimental subjects were divided randomly into four groups. A random number was generated with a computer and combined with the treated drugs randomly. The candidates were randomly given an envelope which had a random number printed outside and the drug information printed inside. Investigators treated the candidate by the indication of drug information inside the envelope. Subjective selection or a changing of drugs was forbidden.

1.4 Drugs Information

TABLE 6
Drugs List

| Drug Name | Manufacture | Size | |
|---|---|---|---|
| NA | | | |
| IFN-α | Kawin Technology | 5 | MIU/Dose |
| GM-CSF | North China | 75 | μg/Dose |
| | Pharmaceutical Group | | |
| HBV VACCINE | North China | 20 | μg/Dose |
| | Pharmaceutical Group | | |

1.5 Immune Therapy Strategy

Group I: Keep oral administration with NA once a day; Group II: Add IFN-αadministration by intramuscular injection with one day interval; Group III: Add GM-CSF administration by intramuscular injection for three continuous days; Group IV: Add intramuscular injection of HBV Vaccine one day after GM-CSF administration.

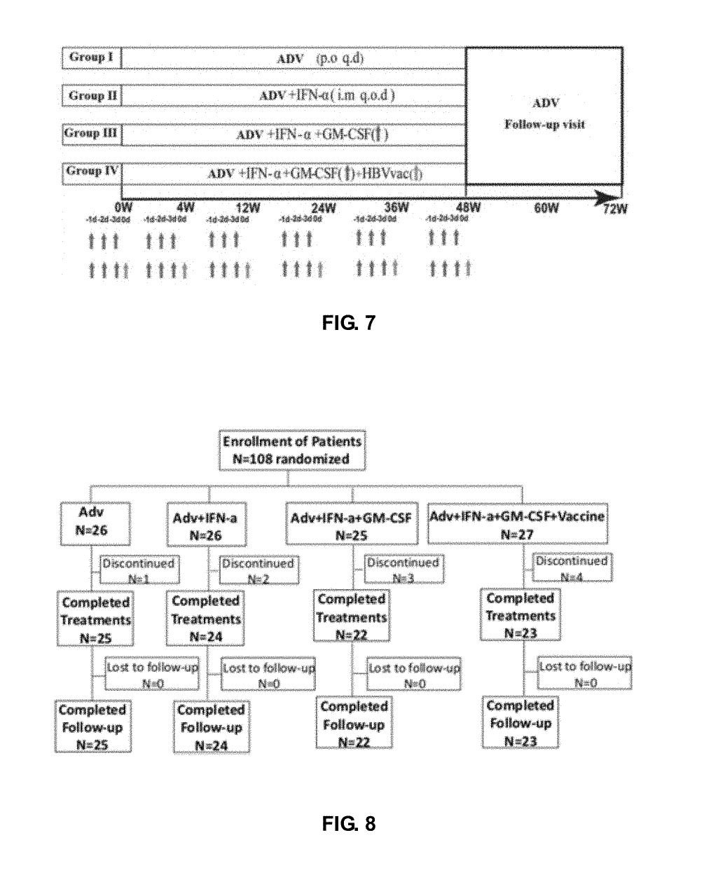

The administration of IFN-α, GM-CSF, and HBV Vaccine will last to week 48, and the ADV administration will last to week 72 with follow-up visit. A sketch of the immune therapy is shown in FIG. 7.

1.6 Halt Criteria

Poor compliance of the experiment subjects; Pregnancy of the experiment subjects; Adverse reaction of the experiment subjects; Quit of the experiment subjects; Death or cannot follow-up visited with the experiment subjects.

1.7 Evaluation of the Curative Effect and the Safety Assessment

1.7.1 The Main Indicator for the Evaluation of the Curative Effect

HBsAg transformed to negative; HBsAg serotype transformed (HBsAg<0.05 IU/ML and HBsAG>10 IU/ML); HBsAg decreased at least 10% of the basement.

1.7.2 Other Indicators for the Evaluation of the Curative Effect

HBV DNA was not detected or the level decreased at least 2 log 10 IU/mL compared with the basement on weeks 12, 24, 36, 48 and 72; The AST and ALT level on weeks 12, 24, 36, 48 and 72; The histological response, with the inflammation and fibrosis should rate under level 1 of the liver on week 48.

1.7.3 Indicators for Safety Assessment

Vital sign; Adverse reaction; Anaphylactic reaction; Clinical data; Physical examination.

1.8 Virology Detection and Biochemical Detection

Routine biochemistry (liver function, renal function); Blood cell analysis; Serum HBV DNA detection; HBVM detection; All the detection were conducted in the central laboratory of Huashan Hospital.

1.9 Clinical Cellular Immune Detection

Cellular detection was conducted in Key Laboratory of molecular virology, Ministry of education, Fudan University.

1.9.1 Reagents

TABLE 7
Reagents List

| Name | Lot | Size | | Manufacture |
|---|---|---|---|---|
| Lymphocyte Separation | 1114546 | 599 | mL | Axis-shield |
| Medium | | | | |
| RPMI-1640 | 11875-093 | 500 | mL | Gibco |
| Fetal Bovine Serum | 10100-147 | 500 | mL | Gbico |
| Penicillin-Streptomycin | BL505A | 100 | mL | Wohong, |
| | | | | Nanjing |
| Anti-Human CD28 | 130-093-375 | 100 | μg/mL | Miltenyi |
| antibody | | | | Biotec |
| Recombinant human IL-2 | AE-200-02 | 100 | μg | Peprotech |
| Phorbol ester | P1585 | 1 | mg | Sigma |
| Lonomycin | 10634 | 2 | mg | Sigma |
| Brefeldin A | 555029 | 1 | mL | BD |
| PBS | SH30256.01 | 500 | mL | Hyclone |
| DMSO | 67-68-5 | 100 | mL | AppliChem |
| 4% PFA | G1101 | 500 | mL | Guge, Wuhan |
| Intracellular Staining | 421002 | 100 | mL | Biolegend |
| Permeabilization | | | | |
| Wash Buffer | | | | |
| FOXP3 Fix/Perm | 421401 | 30 | mL | Biolegend |
| Buffer (4x) | | | | |

1.9.2 Antibodies

TABLE 8
Antibodies List

| | Clone | | Manufac- |
|---|---|---|---|
| Name | number | Isotype | turer |
| APC Anti-human CD4 | OKT4 | Mouse IgG2b, κ | eBioscience |
| FITC Anti-human CD4 | OKT4 | Mouse IgG2b, κ | eBioscience |
| APC Anti-human CD8 | SK1 | Mouse IgG1, κ | eBioscience |
| PE Anti-human IFN-γ | 4S.B3 | Mouse IgG1, κ | eBioscience |
| FITC Anti-human IL-2 | MQ1- | Rat IgG2a, κ | eBioscience |
| | 17H12 | | |
| APC Anti-human FOXP3 | PCH101 | Rat IgG2a, κ | eBioscience |
| PE Anti-human IL-10 | JES3-9D7 | Rat IgG1, κ | eBioscience |
| Brilliant Violet 421 ™ | MAb11 | Mouse IgG1, κ | BioLegend |
| Anti-human TNF-α | | | |
| Brilliant Violet 421 ™ | MP4-25D2 | Rat IgG1, κ | BioLegend |
| Anti-human IL-4 | | | |
| APC/Cy7 Anti-human | BL168 | Mouse IgG1, κ | BioLegend |
| IL-17A | | | |

1.9.3 Peptides

Peptides were synthetized in Shanghai Peptide Biology Co., Ltd., i.e., [symbol] with, a purity of 95%. The sequence information was detailed in the Tables 2-4.

1.9.4 Blood Sampling, PBMC Isolation and T Cell In Vitro Enrichment

1) Blood sampling and transport. Intravenous blood sample was collected with anticoagulant tube and shaken 5 to 10 times slowly. The sample was kept erect and stored at room temperature. The transport may be finished within 2 hours.

2) Sample recording. Four Cryogenic Vials prepared, 3 for storage of plasma and 1 for storage of PBMC. Blood sample was set for centrifuge with 1600 rpm, 10 min, at room temperature. Then plasma was isolated and stored with 1 mL/vial in −80 ° C.

3) Isolation of PBMC. The rest of the sample was mixed with PBS to a volume of 20 mL and carefully transferred into a new tube with lymphocyte separation medium added before. Then the tube was set for centrifuge with 2000 rpm, with both the accelerate rate and decelerate rate at zero, 30 min., at room temperature.

4) Collection of PBMC. Abandon supernatant with 3 to 5mL. Carefully collect the buffy coat cells and transfer into a new 15 mL tube. Add sterile PBS until 15 mL, set for centrifuge with 2000 rpm, with both the accelerate rate and decelerate rate as nine, 5 min., at room temperature.

5) Preparation of 10% FBS-RPMI-1640. Eight mL of 10% FBS-RPMI-1640 was prepared with 8 ul human IL-2 and 8 ul human CD28 added.

6) Detection of HLA-A2. PBMC was staining with anti-HLA-A2 antibody and detected by FACS.

7) Cell seeding and peptide stimulation. PBMC was re-suspended with 10% FBS-RPMI-1640 and seeded into 12-well plate with 1mL/well. Specific peptides were added at the same time with 1 μL per well. The plate was then incubated in 5% CO₂, 37° C.

1.9.5 Fluid Supplement

After five days incubation, 50 μL sample was taken from each well and stored at −80° C. Then added 500 ul 10% FBS-RPMI-1640 contained 0.75 human IL-2, continued for another five days incubator.

1.9.6 Isolation and Storage of the Enriched Cells

1) Preparation of 10% FBS-RPMI-1640 containing CD28 and BFA (Day 10). For one sample 4 ML 10% FBS-RPMI-1640 was added with 5 ul CD28 and 5 ul BFA.

2) Stimulation. After 10 days enrichment, the cells in 12-well plate were transferred into a 1.5 mL EP tube. Centrifuge with 1500 rpm, 3 min. Discard supernatant and re-suspend with FBS-RPMI-1640 prepared in step 1. The suspended cells were then seeded into a 96-well plate, the plate was coated with PMA, N, CMV, S4, S8 and MIX, 90 uL/well with three repetitions. Then the plate was incubated in 5% CO₂, 37° C. for 8 hours.

3) Storage. After 8 hours incubation, the 96-well plate was set for centrifuge with 1500 rpm, 3 min. Discard supernatant and re-suspend with 200 μL cell freezing medium (10% DMSO with 90% FBS). Disassociation by pipetting 3 to 5 times and sealed with parafilm. And then set for gradient cryopreservation with 4° C. for 1 hour, at −20° C. for 40 min and at −80° C. thereafter.

1.9.7 Cell Staining and Detection.

1) Cell recovery. The −80° C. stored 96-well plate was incubated in 37° C. for 20-30 min. Centrifuged with 1600 rpm, 3 min. Discard supernatant and re-suspend with 150 μL PBS.

2) Transfer into another plate. To facilitate the staining, samples were transferred into another 96-well plate, which with was strictly marked before.

3) Cell surface marker staining Panel 1, CD4-APC; Panel 2, CD8-APC; Panel 3, CD4-FITC. The dose of antibody was as the recommended dose in the reagent manual. Plate was centrifuged with 1500 rpm, 3 min. Discard supernatant and re-suspend with staining buffer in 4° C. for 30 min.

4) Termination of the staining. Add 200 μL PBS to terminate the staining and centrifuged with 1600 rpm, 3 min.

5) Cell fixation. Cells were set for fixation with 4% BFA for 10 min. For Treg staining the cells were fixed with Foxp3 Transcription Factor Staining Buffer Kits. After fixation cells were centrifuged with 3000 rpm, 3 min. Discard supernatant and re-suspend with 200 μL PBS, 3000 rpm, 3 min.

6) Intracellular staining. Panel 1/Panel 2, IFN-g-PE, TNF-α-BV421, IL-17-APC/cy7, IL-2-FITC; Panel 3, FOXP3-APC, IL-4-BV421, IL-10-PE. The dose of antibody was at the recommended dose in the reagent manual. Re-suspend the cells with staining buffer in room temperature for 1.5 hour with shake.

7) Termination of the intracellular staining. Add 200 μL PBS to terminate the staining and centrifuged with 1600 rpm, 3 min.

8) FACS detection. The detection was conducted with BD LSR Fortessa (BD Bioscience, USA). The voltage and compensation may be well adjusted. The result data was analyzed with FlowJo v7.6.3, and the results were shown as positive percentage.

1.10 Data Management and Statistical Analysis

1.10.1 Data Input

The data in our experiment was recorded with Case Report Form (CRF). The CRF can be fulfilled on the internet (URL: https://secure.eclinicalos.com/login?null). The data is open for all participated institutions. In order to avoid the disclosure of the personal information, only the name and random number were used for registration.

1.10.2 Data Verification

After all the input of data was completed, a manual verification was then conducted. The administrator may give a report about research progress, may check of the inclusion and exclusion criteria, may check the integrity, may check the data consistency, and may check the logicality of data and the adverse accident.

1.10.3 Statistical Analysis

Statistical analysis was conducted by SPSS v.19.0. Level of HBsAg was shown as logio. Cochran-Mantel-Haensel (CMH) was used for the comparison analysis among groups. If P<0.05 there is significant differences among the four groups, and then F-test was applied for analysis between two groups. Data in the same group but with different time point were analyzed by a paired t-test. Data with different groups were analyzed with ANOVO. A Pearson correlation analysis was used to compare the correlation between two different indicators. α=0.05 was used for two-tailed significant difference test, P≤0.05 meant that there was significant difference between two groups.

2. The Experimental Results

2.1 Baseline characteristics of subjects. The center recruited 104 patients with chronic hepatitis B, and the patients were randomly assigned to 4 experimental groups. After the elimination of lost follow-ups (withdraw or residence resettlement), 94 patients (90.4%,94/104) completed the trial, 25 for ADV Group, 24 for ADV+IFN-αGroup, 22 for ADV+IFN-α+GM-CSF Group, and 23 for ADV+IFN-α+GM-CSF +the vaccine group, representing 26.6%, 25.5%, 23.4%, and 24.5% of the total number of trials completed (see FIG. 8). After unblind, except for gender (the male ratio in the ADV group was lower than in the other three groups), the baseline data for the four groups was not statistically significant and comparable (p>0.05) (see Table 9).

TABLE 9
Baseline data for clinical trial subjects

| | | | | | IFN-α + | IFN-α + ADV + | |
|---|---|---|---|---|---|---|---|
| | | | | IFN-α + | ADV + | GM-CSF + | |
| | | Total Patients | ADV | ADV | GM-CSF | VACCINE | |
| Characteristic | | (n = 94) | (n = 25) | (n = 24) | (n = 22) | (n = 23) | P value |
| Age (yrs) | Median (SD) | 42.3(10.8) | 44.3(11.2) | 40.5(10.2) | 44.3(9.9) | 40.0(11.7) | 0.685 |
| Male | No. (%) | 82(84.5) | 18(72.0) | 24(100.0) | 20(90.9) | 20(87.0) | 0.042 |
| HbsAg (log₁₀ IU/mL) | Median (SD) | 2.52(0.88) | 2.83(0.76) | 2.38(1.08) | 2.56(0.73) | 2.26(0.83) | 0.178 |
| AST (U/L) | Median (SD) | 24.1(10.3) | 23.8(7.1) | 22.9(8.3) | 26.4(16.4) | 23.6(6.6) | 0.674 |
| ALT (U/L) | Median (SD) | 31.3(16.8) | 27.3(15.2) | 34.4(18.9) | 30.0(12.3) | 34.1(19.9) | 0.087 |
| WBC (×10⁹/L) | Median (SD) | 5.9(1.7) | 5.7(1.8) | 6.0(1.9) | 6.1(1.69) | 5.7(1.3) | 0.790 |
| HLA-A2 | Positive (%) | 50(53.2) | 14(56.0) | 13(54.2) | 11(50.0) | 12(52.2) | 0.992 |

2.2 Serum HBsAg and ALT Kinetics in Subjects

In the ADV Group, no HBsAg transformation nor sero-conversion were observed throughout the 48-week treatment and 72-week follow-up, and ALT level did not change significantly either.

In Adv+IFN-αGroup, at the 24th week, 3 patients (i.e., 3/24) showed HBsAg transformation, accounting for 12.5%. Until the end of the 48 week treatment and the end of the 72 week follow-up, no more patients showed HBsAg decrease, and no HBsAg sero-conversion patients were observed. Serum ALT trend into decrease after first elevation in the treatment process, and peaked at 24th week compared to baseline. Further, the difference was statistically significant (35.1±3.9 IU/L vs 56.8±8.2 IU/L, p<0.05). ALT level dropped to baseline at the end of the treatment of 48 weeks, and was equivalent to the baseline through the follow-up.

In the ADV+IFN-α+GM-CSF Group, at the 24th week, 6 patients (i.e., 6/22) showed HBsAg transformation, accounting for 27.3%. Until the end of 72 week follow-up, 1 out of these 6 patients showed HBsAg clearance (<0.07 IU/L), without sero-conversion. Serum ALT also showed a tendency to rise first and then decrease, reaching the highest peak in the 12th week, with statistically significance from the baseline data (29.3±2.6 IU/L vs 47.2±6.3 IU/L, p<0.05). Again, at 48 weeks, the baseline level was reached until 72 weeks.

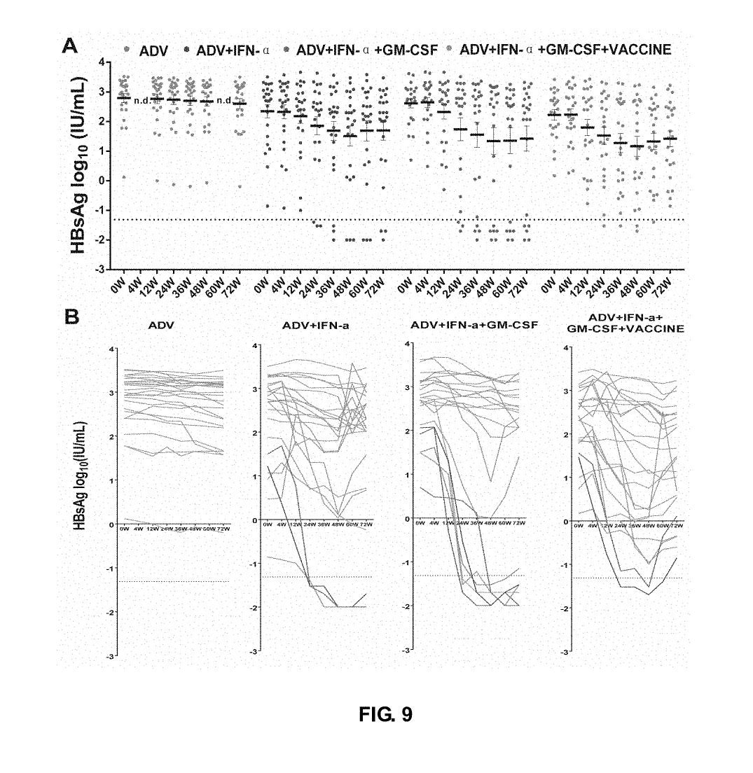

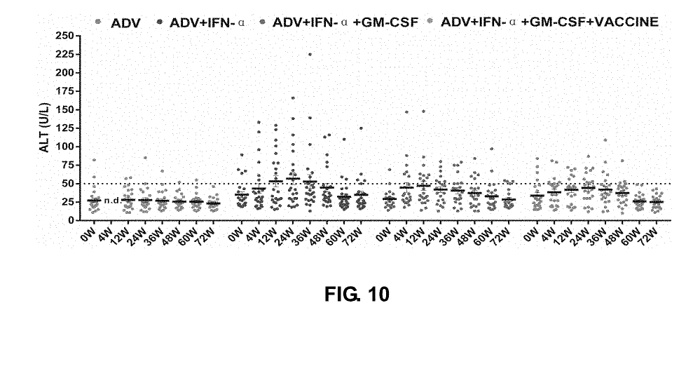

In ADV+IFN-α+GM-CSF+vaccine Group, 1 patient in the 24th week showed HBsAg decrease, and 1 patient showed HBsAg sero-conversion (HBsAb for 692 IU/mL, with HBsAg for 7.2 IU/L). At the end of the 48 week treatment, 2 showed HBsAg serological transformation, 3 had HBsAg sero-conversion (HBsAb 90.0 IU/mL, 24.8 IU/mL and 433.9 IU/mL, respectively). At the end of follow-up, HBsAg were all less than 10 IU/L, HBsAb were 15.7 IU/mL, 15.7 IU/mL and 222.7 IU/mL for above 3 patients. Serum ALT again fluctuated, consistent with the second and third groups. The first elevation was lower, highest in the 24th week, not statistically significant comparing with baseline (33.8±4.1 IU/L vs 44.3±4.1 IU/L, p>0.05). Again, at 48 weeks, the baseline level was reached until 72 weeks (see FIGS. 9 and 10)

2.3 Clinical and Immunological Evaluation.

Antigen specific cellular immunity may play an important role in chronic HBV treatment. In order to understand the dynamic changes of cellular immunity during our treatment course, samples were collected 8 repeated times, including 6 times in the treatment process (before the first treatment (i.e., at 0 weeks), 4 weeks (1th Group not Blood collection), 12 weeks, 24 weeks, 36 weeks, and 48 weeks) and 2 times during the follow-up process (60 weeks (the 1st group did not collect blood) and 72 weeks). Peripheral Blood mononuclear cells (PBMC) were isolated and re-stimulated with HBV-specific polypeptide for 10-day enrichment. After the cell was collected and re-stimulated by HBV-specific polypeptide for 8 hours, the levels of cytokines in CD4+ and CD8⁺ T lymphocytes were detected by flow cytometry. IL-2, IL-4, IL-10, IL-17a, Foxp3, IFN-γ and TNF-α were analyzed in CD4⁺ T cells, whereas IL-2, IL-17a, IFN-γ and TNF-α were analyzed in CD8⁺ T lymphocyte.

2.3.1 Quality Control of Clinical Immunology Ttest.

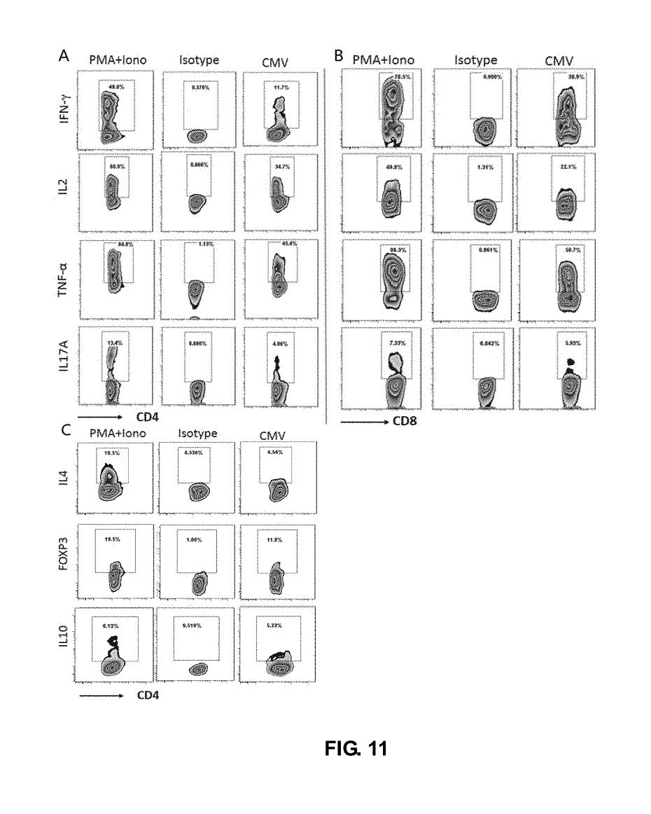

First, to ensure the accuracy and robustness, positive controls for re-stimulation were added, such as PMA/Ionomycin, and human cytomegalovirus polypeptide (CMV, PP65). As shown in FIG. 11, both PMA/Ionomycin and CMV peptide stimulated cytokine release and the expression of transcription factor Foxp3.

2.3.2 Expression Level of Regulatory Factors in Treg Cells.

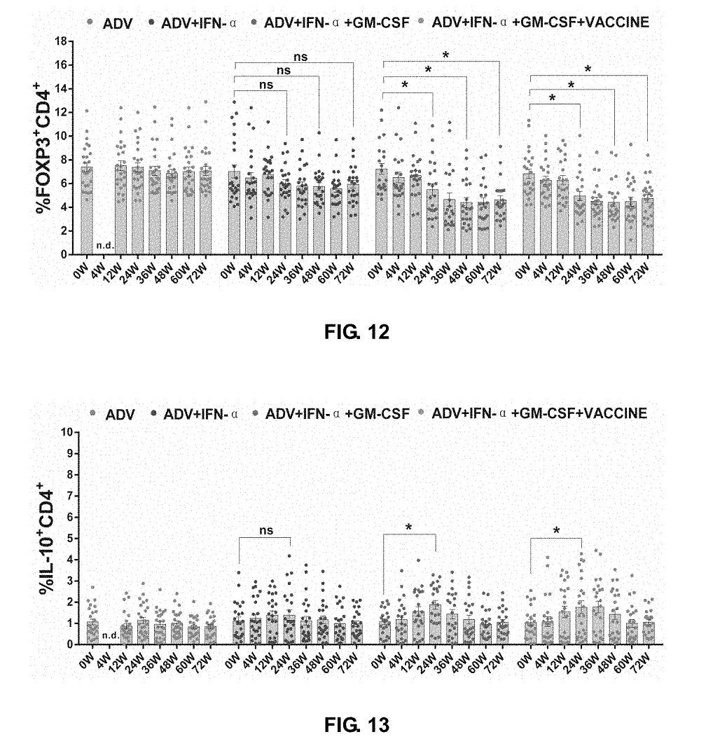

Treatment of chronic hepatitis B may be generally difficult because immune tolerance induced by HBV, and breaking the immune tolerance is the key. Regulatory T cells (Treg) may play an important role in maintaining the immune tolerance and Foxp3 is the most important transcription factor in Treg. Therefore, Foxp3 expression dynamics in CD4⁺ T lymphocytes in four groups was analyzed. As shown in FIG. 12, Foxp3 in CD4⁺ T cell from 0 weeks to 72 weeks was not significantly changed in the ADV group. In the ADV+IFN-α group, Foxp3 showed a first downward trend, then returned to baseline level after treatment at the 48th week. Both ADV+IFN-α+GM-CSF Group and ADV+IFN-α+GM-CSF+vaccine group were similar than that in the 24th week, Foxp3 decreased significantly (p<0.05). After treatment withdrawal, Foxp3 expression increased, but did not revive to the baseline level, and the difference was statistically significant (p<0.05).

IL-10 may be a key inhibitory cytokine secreted by Treg, therefore IL-10 secretion dynamics in CD4⁻ T cell were analyzed and are shown in FIG. 13. In the ADV group, no significant change was observed. In the ADV+IFN-α group, IL-10 showed a trend of ascending first and then descending (p>0.05). Again, both the ADV+IFN-α+GM-CSF Group and the ADV+IFN-α+GM-CSF+vaccine group were similar in the 24th week, in that IL-10 increased significantly (p<0.05), followed by a downward trend up to 72 weeks to the baseline level.

2.3.3 Th1 Cytokines Stimulated by S-Peptide Pool in CD4⁺T cells.

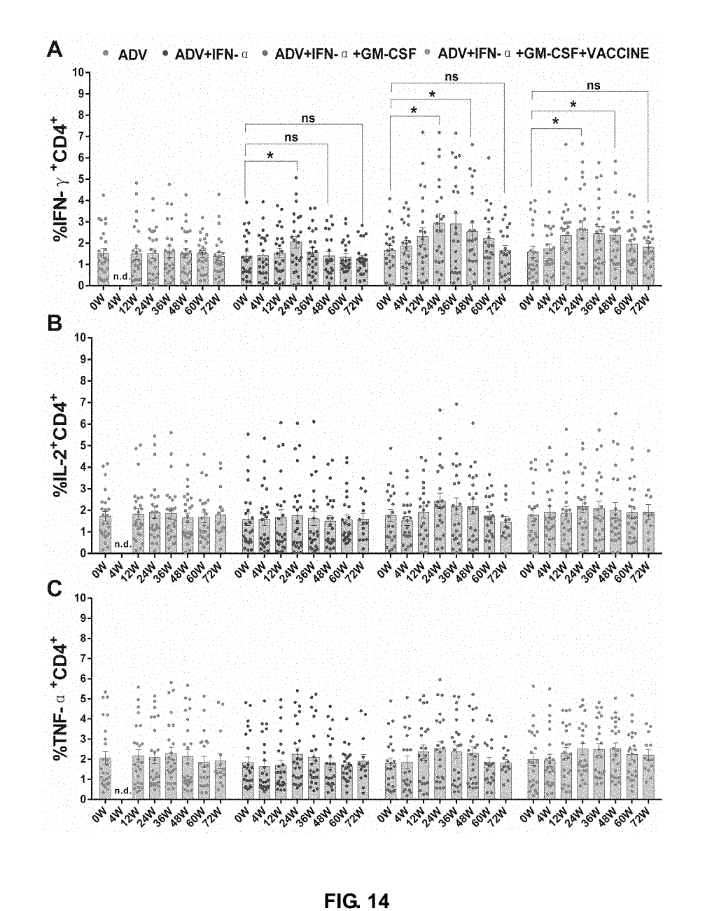

Th1 cell immunization plays an important auxiliary role in chronic hepatitis B treatment, in addition to breaking immune tolerance. Therefore, the PBMC after the stimulation of the S-polypeptide pool for CD4⁺ T cells was analyzed. Three representative cytokines IFN-γ, IL-2, TNF-α were analyzed and plotted in dynamics. IFN-γ secretion in CD4⁺ T cells in the ADV group showed no significant difference from 0 to 72 weeks. In the ADV+IFN-α group, IFN-γ showed a trend of ascending and descending from 0 weeks to 72 weeks (p>0.05). In both the ADV+IFN-α+GM-CSF Group and the ADV+IFN-α+GM-CSF+vaccine group, IFN-γ expression in CD4⁺T cells increased significantly (p<0.05) and then decreased to the baseline level at 72 weeks, as shown in FIG. 14. IL-2 and TNF-α showed no significant difference in the ADV group. In the ADV+IFN-α group, the ADV+IFN-α+GM-CSF group, and the ADV+IFN-α+GM-CSF+Vaccine group, they both had a slight increase in the course of treatment without significance (p>0.05), and then gradually reverted to the baseline level, as shown in FIG. 14.

2.3.4 Th2 Cytokines Stimulated by S-Peptide in CD4⁺Tcells.

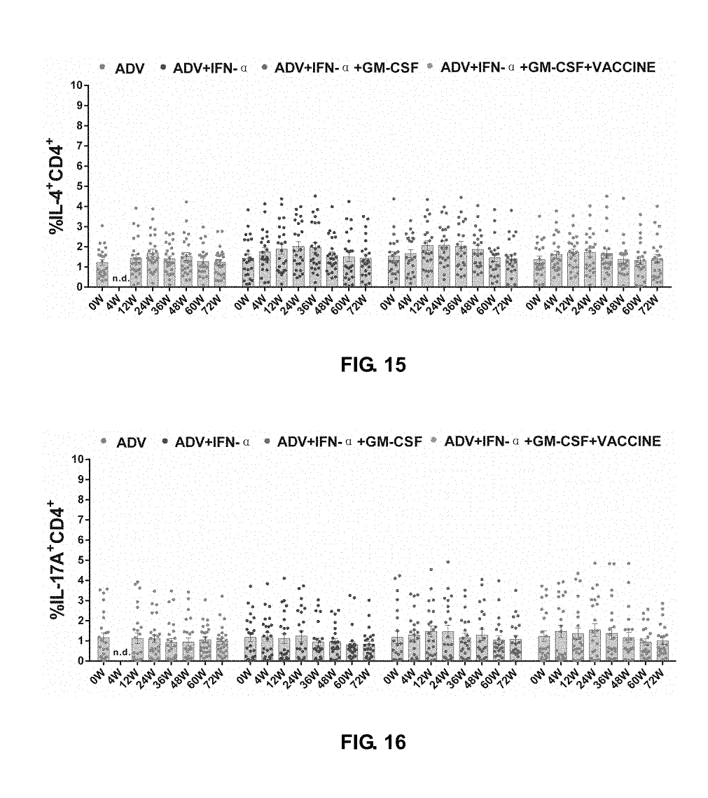

Th2 cells mainly function by helping B cells to produce antibodies. In Result 3.2, it has been found that the ADV+IFN-α+GM-CSF+vaccine group produced an Anti-HBsAg antibody, whereas the remaining three groups did not. Here, the Th2 cells re-stimulated by the S-polypeptide pool with representative cytokine IL-4 dynamics was further analyzed. IL-4 showed no difference in the ADV group. In the ADV+IFN-α group, the ADV+IFN-α+GM-CSF group and the ADV+IFN-α+GM-CSF+vaccine group, IL-4 had a slight increase without significance (p>0.05), and then gradually reverted to the baseline level, as shown in FIG. 15.

2.3.5 Th17 Cytokines Stimulated by S-Peptide in CD4⁺T Cells.

The expression levels of TH17 cells in 0, 4, 12, 24, 36, 48, 60, and 72 weeks after the stimulation of the CD4⁺Tcell by the PBMC peptide pool in four treatment groups were studied. There was no significant change in the IL-17A in the ADV group. In the ADV+IFN-α group, the ADV+IFN-α+GM-CSF group, and the ADV+IFN-α+GM-CSF+vaccine group there was slight elevation in the course of treatment without significance (p>0.05) and gradually dropped after 48 weeks of treatment, back to the baseline level at 72 weeks, as shown in FIG. 16.

2.3.6 TC1 Cytokines Stimulated by S-Peptide in CD8⁺T Cells.

TC1 cells strength may have a direct effect on HBV removal. Three representative cytokines for TC1, IFN-γ, IL-2 and TNF-α dynamics, after (HLA-A2⁺or HLA-A2⁻) S polypeptide re-stimulation in CD8⁺ T cells were analyzed and plotted.

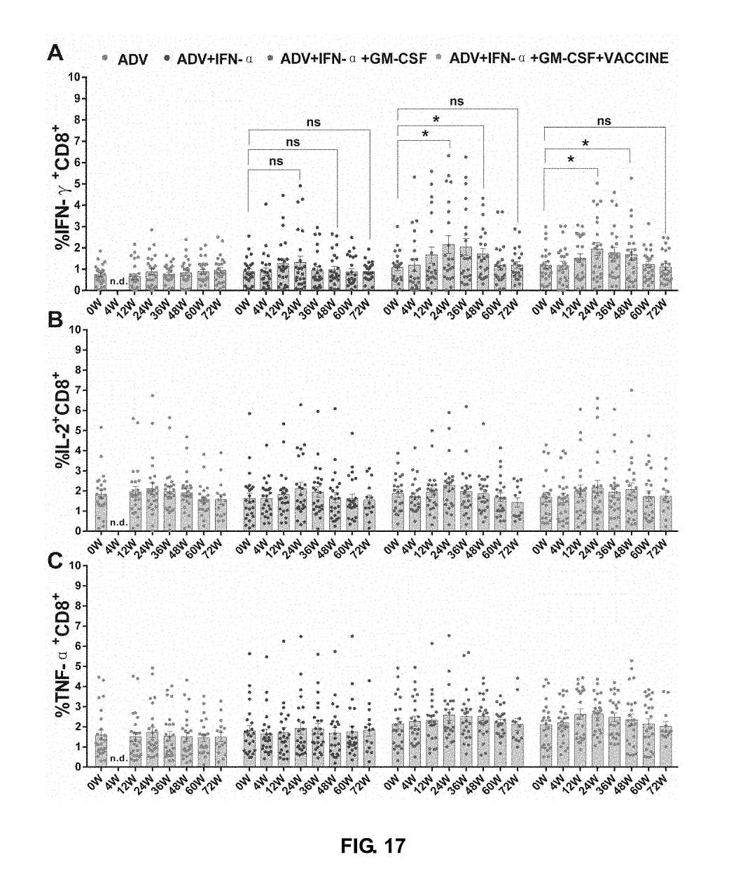

IFN-γ secreted by CD8⁺ T cells showed no difference in the ADV group. The ADV+IFN-α group showed a trend of ascending and descending from 0 weeks to 72 weeks, without significance (p>0.05). In the ADV+IFN-α+GM-CSF group and the ADV+IFN-α+GM-CSF+vaccine group, the expression of CD8⁺ T cell IFN-γ increased significantly (p<0.05) in 24 week and then decreased until 72 weeks to the baseline level, as shown in FIG. 17.

No significant difference was found in the IL-2 and the tnf-α secreted by CD8⁺T cells in ADV group. In the ADV+IFN-α group, the ADV+IFN-α+GM-CSF group, and the ADV+IFN-α+GM-CSF+Vaccine group both cytokines had a slightly higher high level in the course of treatment without significance (p>0.05), and then gradually reverted to the baseline level, as shown in FIG. 17.

2.3.7 TC17 Cytokines Stimulated by S-Peptide in CD8⁺T Cells.

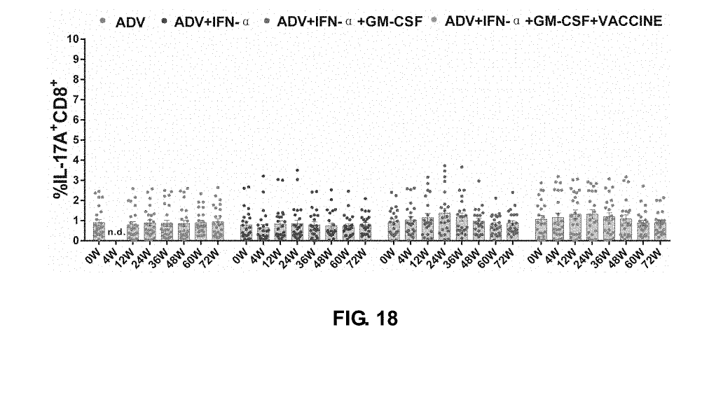

TC17 cells after the stimulation with (HLA-A2⁺ or HLA-A2⁻) S polypeptide pool was studied. Again, IL-17A showed no significance in the ADV group and the ADV+IFN-α groups. In the ADV+IFN-α+GM-CSF group and the ADV+IFN-α+GM-CSF +vaccine group, IL-17A was slightly elevated without significance (p>0.05) and was gradually dropped after 48 weeks of treatment, back to baseline level at 72 weeks, as shown in FIG. 18.

2.3.8 TC1 Cytokines in CD8⁺T Cells After (S+c+pol) Restimulation.

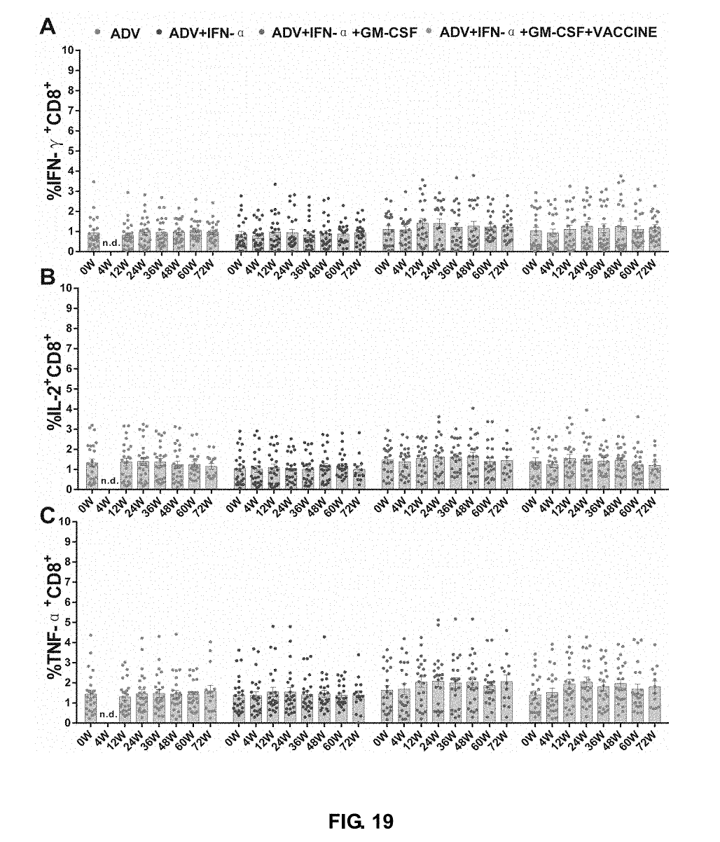

The HBsAg protein vaccine was used in clinical trials to further understand the responsiveness of the core antigen (C) and polymerase (Pol) in our treatment. Thus, CD8⁺T cells after stimulation of the h1a-a2⁺ (S+c+pol) mixed peptides were analyzed. IFN-γ (see FIG. 19), IL-2 (see FIG. 19), TNF-α (see FIG. 19) secreted by CD8⁺T cells showed no statistical significance (p>0.05).

2.3.9 TC17 Cytokines in CD8⁺T Cells After (S+c+pol) Re-Stimulation.

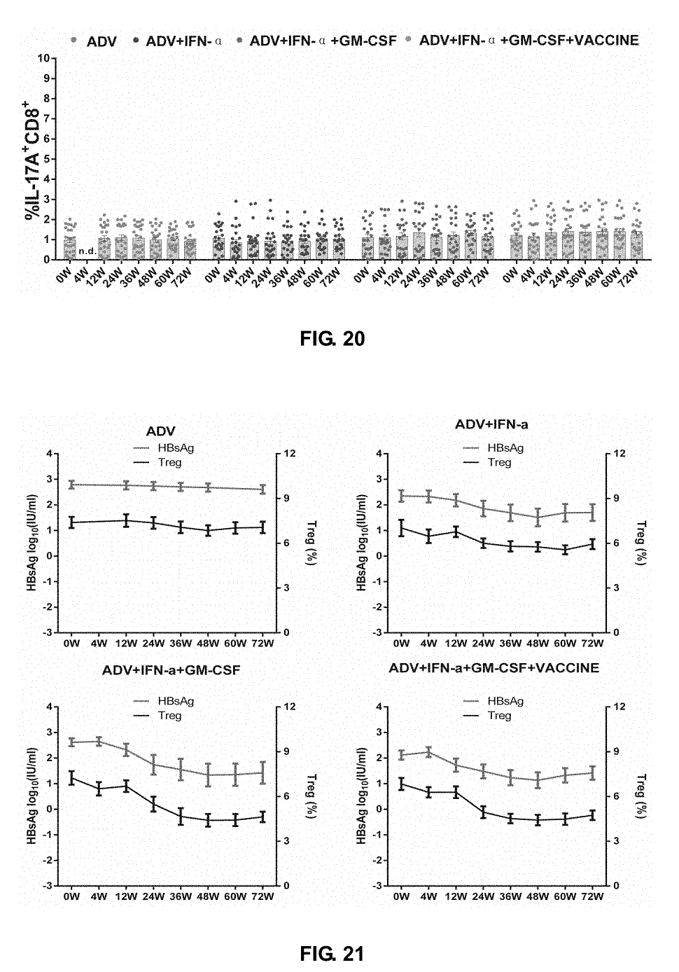

Next, the expression levels of Tc17 cells in 0, 4, 12, 24, 36, 48, 60, 72 weeks after restimulation by h1a-a2⁻ (S+c+pol) mixed peptide were studied. For the IL-17a of CD8⁺T cells, there was no statistical significance (p>0.05), as shown in FIG. 20.

2.3.10 Dynamics and Correlation Between HBgAg and Treg.

HBsAg is known to contribute to immune tolerance. Therefore, to understand whether the level of HBsAg affects Treg level after treatment, the dynamics of HBsAg and Treg in the course of CHB was first analyzed, and the results are shown in FIG. 21. In the ADV group, the HBsAg did not decrease significantly in the whole treatment, and Treg did not change significantly either. In the ADV+IFN-α group, the ADV+IFN-α+GM-CSF group, and the ADV+IFN-α+GM-CSF+vaccine group, HBsAg showed a decrease and then a rise, and the trend of Treg followed the same trend.

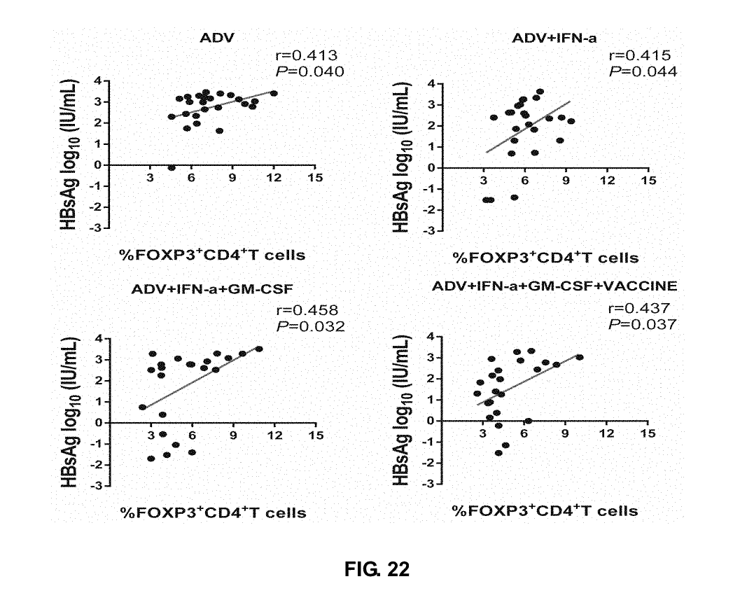

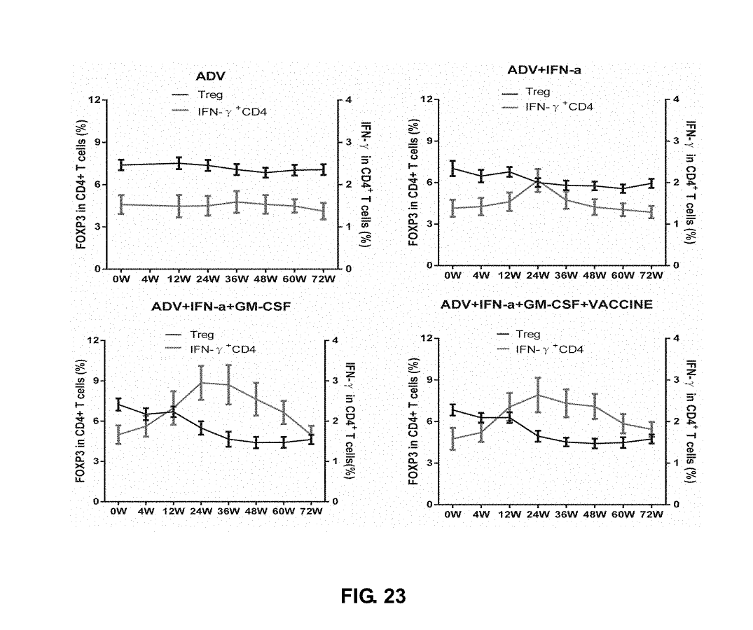

According to the previous results, the HBsAg in the ADV+IFN-α group, the ADV+IFN-α+GM-CSF group and the ADV+IFN-α+GM-CSF+vaccine group decreased most obviously at 24 weeks, at the same time the Foxp3 expression was the most obvious. Therefore, to explore this correlation, the Pearson correlation of HBsAg level and Treg level in 24 week was analyzed, and the results are shown in FIG. 22. In the ADV group, there was a positive correlation between HBsAg and Treg (p<0.05, r=0.413). In the ADV+IFN-α group, positive correlation between HBsAg and Treg was found (p<0.05, r=0.466). In the ADV+IFN-α+GM-CSF group, there was a positive correlation between HBsAg and Treg (p<0.05, r=0.469). Finally, in the ADV+IFN-α+GM-CSF+vaccine group, a positive correlation was found between HBsAg and Treg (p<0.05, r=0.437). From the correlation between the above HBsAg and Treg, it can be deduced that HBsAg is an important factor affecting immune tolerance.

2.3.11 Dynamics and Correlation Between Foxp3 and IFN-γ.

From the previous results, Foxp3 in CD4⁺ T cells showed a down-up trend whereas IFN-γ in CD4⁺ T cells showed opposite up-down trend. As Foxp3 was the inhibitory and IFN-γ was the inflammatory, a correlation between these two in CD4⁺ T cells was explored, as shown in FIG. 23. In the ADV Group, Foxp3 showed no significant fluctuation, neither was IFN-γ. In the ADV+IFN-α group, Foxp3 first declined and then increased, whereas IFN-γ secretion showed a rise then a decline, especially at 24 week. In the ADV+IFN-α+GM-CSF group and the ADV+IFN-α+GM-CSF+vaccine group, Foxp3 first declined after treatment and then increased, interestingly, IFN-γ showed obvious rise after treatment.

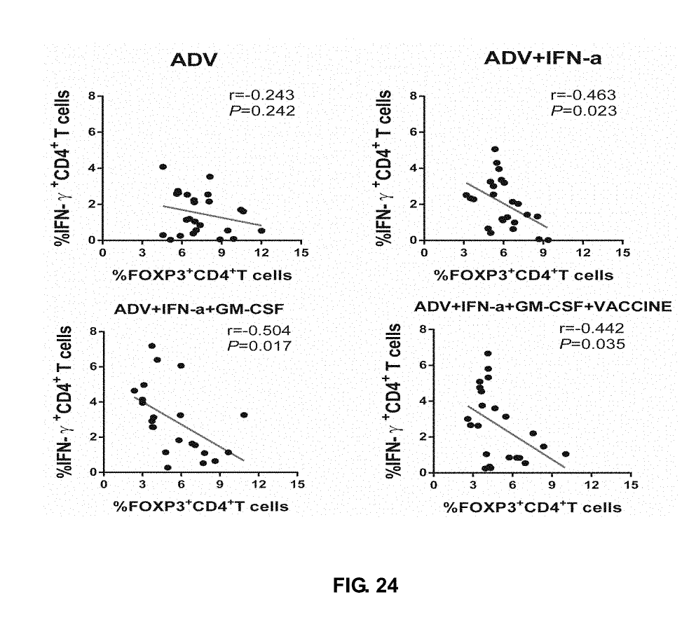

According to the previous results, the Foxp3⁺CD4⁺ T cells decreased in the ADV+IFN-α group, the ADV+IFN-α+GM-CSF group, and the ADV+IFN-α+GM-CSF+vaccine group at 24 weeks, while the IFN-γ⁺CD4⁺T cells rose most obviously. In order to further understand the relationship between Foxp3⁻CD4⁺T cells and IFN-γ⁺CD4⁺T cells, the Pearson correlation at 24 weeks was analyzed and is shown in FIG. 24.

In the ADV group, no correlation between the Foxp3 level and the IFN-γ level in CD4⁺ T cells was observed (p>0.05). In the ADV+IFN-α group, Foxp3 levels were negatively correlated with IFN-γ (p<0.05; r=−0.463). In the ADV+IFN-α+GM-CSF group, Foxp3 and IFN-γ were negatively correlated (p<0.05, r=−0.504). In the ADV+IFN-α+GM-CSF+vaccine Foxp3 was also negatively correlated with IFN-γ (0.05, r=0.442). From the correlation between the Foxp3 level and the IFN-γ level, it may be deduced that IFN-γ⁺CD4⁺ T cells may be one of the important cells to break the immune tolerance.

2.3.12 Dynamics and correlation between Foxp3 in CD4⁺ T cells and IFN-γ in CD8⁺ T cells.

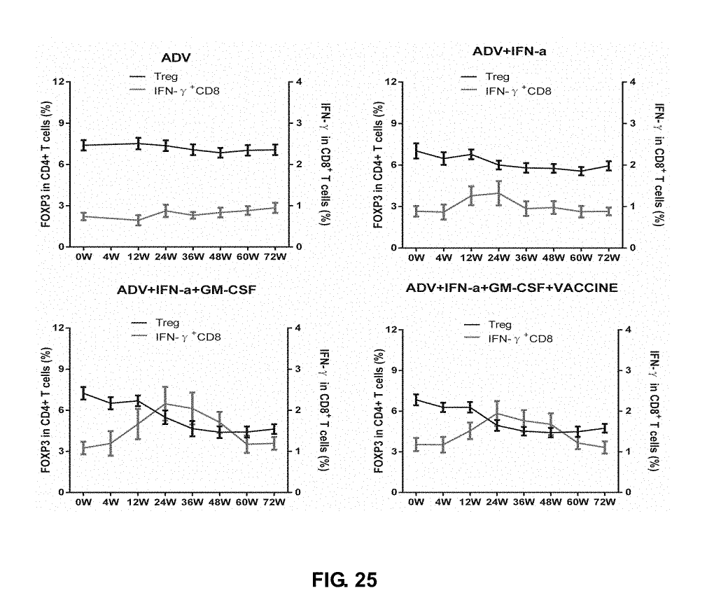

From previous results, Foxp3 in CD4⁺ T cells exhibited an up-down trend, while IFN-γ in CD8⁺T cells showed the opposite down-up trend. As IFN-γ⁺CD8⁺ T cells played a key role in the process of HBV removal, the dynamics and correlation between Foxp3 in CD4⁺ T cells and IFN-γ in CD8⁺T cells was analyzed, as shown in FIG. 25.

In the ADV group, Foxp3 did not show significant fluctuation, neither did IFN-γ secretion. In the ADV+IFN-α group, Foxp3 showed a down-up trend, and IFN-γ secretion showed an up-down trend, especially at week 24. In the ADV+IFN-α+GM-CSF group and the ADV+IFN-α+GM-CSF+vaccine group, Foxp3 first declined and then increased, in the opposite, IFN-γ first increased, then declined.

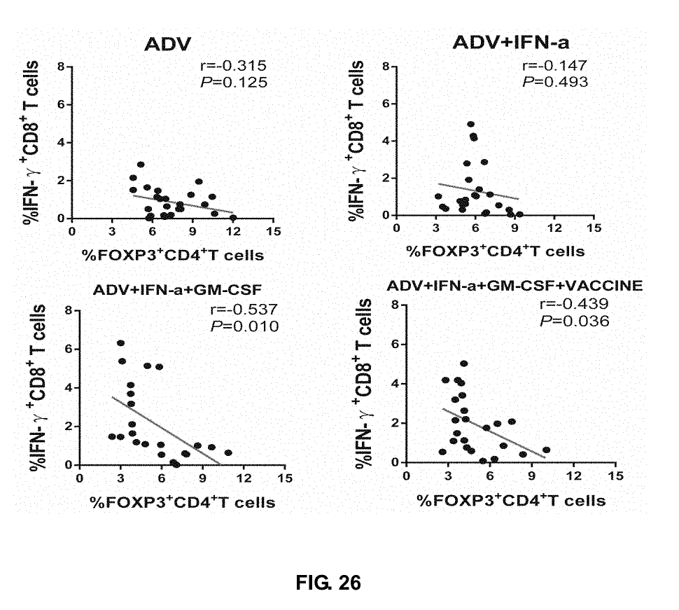

According to previous results, the Foxp3⁻CD4⁺T cells had the most obvious decline at week 24, in the ADV+IFN-α group, the ADV+IFN-α+GM-CSF group, and the ADV+IFN-α+GM-CSF+vaccine group. In order to further understand the relationship between Foxp3⁺CD4⁺T cells and IFN-γ⁺CD4⁺T cells, the Pearson correlation at week 24 was analyzed, as shown in FIG. 26.

There was no correlation between Foxp3 in CD4⁺ T cells and IFN-γ in CD8⁺T cells in the ADV group and the ADV+IFN-α group (p>0.05). In the ADV+IFN-α+GM-CSF group, Foxp3 inCD4⁺T cells and IFN-γ in CD8⁺ T cells were negatively correlated (p<0.05, r=-0.537). In the ADV+IFN-α+GM-CSF+vaccine group, Foxp3 in CD4⁺ T cells were negatively correlated with CD8⁻T cells (p<0.05, r=0.439). The correlation between Foxp3 levels in the above CD4⁺T cells and IFN-γ levels in CD8⁺T cells shows that ADV+IFN-α+GM-CSF and ADV+IFN-α+GM-CSF groups may promote the IFN-γ production in CD8+ T cells, in turn contributing to breaking the immune tolerance, the final elimination of HBV.

2.3.13 T-Lymphocyte Subset's Dynamics During Treatment.

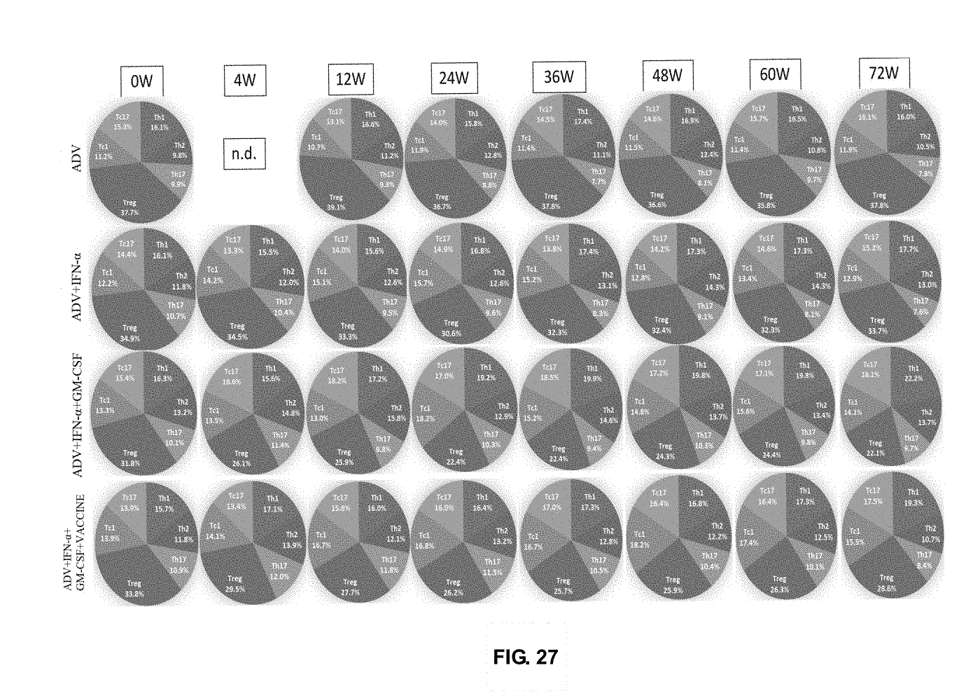

From the previous results, a T cell responds heterogeneously. In order to intuitively understand the dynamic changes of different T cell subsets, IFN-γ⁺ CD4⁺ T cells were assigned into the Th1, IL-4⁺ CD4⁺ T cells as TH2, IL-17a producing CD4⁺ T cells as TH17, Foxp3⁺CD4⁺ T cells Treg, and the CD8⁺ T cells secreting different cytokines were classified as TC1 and TH17 by the same method. Next, a proportion of subsets of cells in the total T cell were analyzed and plotted, as shown in FIG. 27.

In the ADV Group, the baseline of Treg cells was 37.7%, at 48 weeks was 25.8%, from 0 weeks to the end of treatment 48 weeks were basically unchanged, followed by 72 weeks for 37.8%. Similarly, for TH1, TH2, TH17, TC1, and Tc17 cell proportions, there were no significant changes from 0 weeks to 48 weeks to 72 weeks.

In the ADV+IFN-α group, the baseline of Treg cells was 34.9%, at 24 weeks was 30.6%, 48 weeks was 32.4%, the follow-up endpoint at 72 weeks was 33.7%, and the baseline to the follow-up end decreased 1.2%, and for Th1, TH2, TH17, Tc1, and Tc17 cells, with small change less than 3%.

In the ADV+IFN-α+GM-CSF group, the baseline of Treg cells was 31.8%, at 24 weeks to 22.4%, decreased by 9.4%, 48 weeks, slightly up to 24.3%, followed by 72 weeks at the end of the period, from the baseline to the end of the follow-up drop of 22.1%. The proportion of Th1 cells increased from 16.3% in the baseline to 22.2% at the end of the follow-up period, increasing the proportion of 5.9%. TC1 cells from 13.3% to 24 weeks from the baseline to 18.2%, increasing 5.9%, and then continuing to decline to 14.1% at the end of the follow-up period for TH2. In the TH17 and TC17 cells, with small change less than 3%.

In the ADV+IFN-α+GM-CSF+vaccine group, the baseline of Treg cells was 33.8%, at 24 weeks, to 26.2%, down 7.6%, 48 weeks, 25.9%, followed by 72 weeks, and 28.6% from baseline to the end of follow-up. The proportion of Th1 cells increased from 15.7% in the baseline to 19.3% at the end of the follow-up period, increasing the proportion of 3.6%, TC1 cells from 13.9% to 24 weeks from the baseline to 16.8%, increasing 2.9%, and then continuing to decline to 15.5% at the end of the follow-up period, for TH2, In the TH17 and TC17 cells, with small change less than 3%.

2.3.14 Dynamics of Th1\Th2\Th17\/Treg proportion in CD4⁺ T cells and Th1/Th2 ratio.

In order to further obtain the effect of different therapies on cellular immunity, the TH1/Treg, Th2/Treg, Th17/Treg, and Th1/th2 was analyzed and plotted.

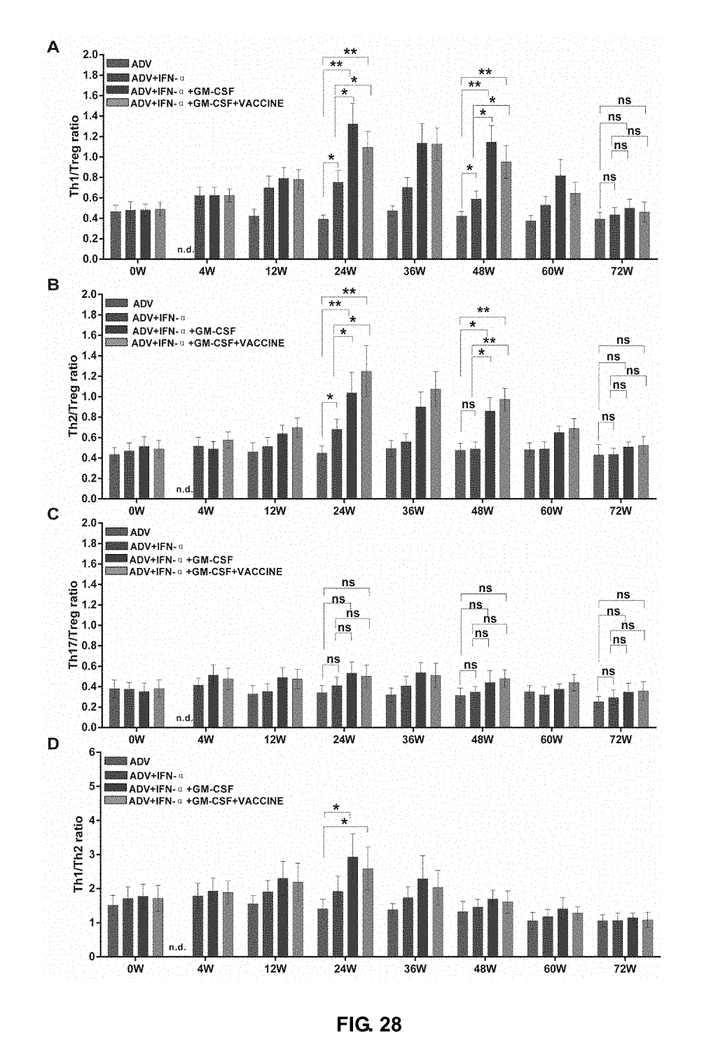

For the TH1/Treg ratio, there was no difference among groups at baseline. At 24 weeks, the ADV+IFN-α group had a significantly higher TH1/Treg ratio than the ADV group (p<0.05). In the ADV+IFN-α+GM-CSF group and the ADV+IFN-α+GM-CSF, the TH1/Treg ratio was significantly higher than that in the ADV Group (p<0.01), while the ADV+IFN-α+GM-CSF and the ADV+IFN-α+GM-CSF +vaccine groups were higher than the ADV+IFN-α group (p<0.05). At the end of treatment at 48 weeks, the TH1/Treg ratio of each group was the same at 24 weeks, whereas at 72 weeks after the end of the group, there was no statistically significant difference between the TH1/Treg ratios (p>0.05), see FIG. 28.

For the Th2/Treg ratio, there was no difference among groups at baseline. At 24 weeks, the ADV+IFN-α group had a significantly higher Th2/Treg ratio than the ADV group (p<0.05). In the ADV+IFN-α+GM-CSF group and the ADV+IFN-α+GM-CSF group, the Th2/Treg ratio was significantly higher than that in the ADV group (p<0.01), while the ADV+IFN-α+GM-CSF group and the ADV+IFN-α+GM-CSF +vaccine group were higher than the ADV+IFN-α group (p<0.05). At the end of treatment at 48 weeks, there was no difference in Th2/Treg between the ADV group and the ADV+IFN-α group (p>0.05), the ADV+IFN-α+GM-CSF group was higher than the ADV group and the ADV+IFN-α group. There was a statistically significant difference (p<0.05), and the ratio of Th2/Treg in ADV+IFN-α+GM-CSF+vaccine group was significantly higher than that of the ADV group and the ADV+IFN-α group, with statistically significant difference (p<0.01), the Th2/Treg proportion of each group was not statistically significant (p>0.05) at the end of 72 weeks of follow-up, see FIG. 28.

For Th17/Treg ratio, from 0 weeks to 48 weeks, up to 72 weeks of follow-up, there was no difference between four treatment groups (p>0.05), see FIG. 28. For the th1/th2 ratio, at 0 weeks, there was no difference between the four groups, at 24 weeks, the ADV+IFN-α+GM-CSF group and the ADV+IFN-α+GM-CSF+vaccine group was higher than the ADV group, and the difference was statistically significant (p<0.05), see FIG. 28.

3. Discussion

Hepatitis B Therapeutic vaccines are considered a potential immunotherapy for chronic hepatitis B, however, development of hepatitis B therapeutic vaccines is challenging, especially with regard to breaking the immune tolerance of hepatitis B, and stimulating the body against HBV specific cellular immune function is limited. One of the possibilities is the lack of an appropriate adjuvant. Here, this clinical study has two main tasks: first, to explore the efficacy and feasibility of GM-CSF as an adjuvant for a Hepatitis B therapeutic vaccine in the treatment of patients with chronic hepatitis B, and second, to provide a clinical immunology experimental technical plan and an accompanying diagnosis basis for the evaluation of HBV interventions.

Curing hepatitis B may be rooted in HBsAg transformation and sero-convertion. The natural conversion rate is annual 1-2%. Even polyethylene glycol interferon alpha and nucleoside (acid) antiviral drugs combined treatment was administrated, conversion rate is as low as 5% per year. According to data in this clinical trial, after 48 weeks treatment, sero-conversion rate of HBSAG and/or the conversion rate of the ADV+IFN-α+GM-CSF+vaccine group was 15% (the data unpublished). The serum conversion rate and/or conversion rate of HBSAG in the ADV+IFN-α+GM-CSF+vaccine group was 13%, which was 3 times more than any other therapies, thus a 3×GM-CSF+vaccine therapy had obvious advantages in treating chronic hepatitis B patients.

Regulatory T cells (Regulatory T cell, Treg) are one of the important causes of immune tolerance in HBV infection. Shrivastava and other studies showed that, compared with HBsAg negative and healthy people, the frequency of Foxp3⁺ regulated T cells increased significantly in HBsAg-positive neonates, suggesting that the level of HBSAG was positively correlated with Treg level [26]. The decline of Treg is considered an important indicator to break immune tolerance. In the results described herein, after ADV+IFN-α treatment, the HBSAG decreased and the Treg frequency was also associated with the HBSAG, which was consistent with other experimental results. Compared with the ADV+IFN-α group, the Treg frequency of the ADV+IFN-α+GM-CSF group and the ADV+IFN-α+GM-CSF+vaccine group was significantly lower than that in the baseline (p<0.05), which showed that the 3×GM-CSF+Vaccine can more effectively break the immune tolerance state of CHB patients.

Specific immune activation is an important mechanism for the removal of HBV, including the enhancement of helper T cell activation and the increase of CTL ability and humoral immunity. In this experiment, the specific cellular immunity of the organism was basically unchanged after treatment with nucleoside (acid) analogues. After the addition of IFN-α, the body specific cellular immunity did not have a significant enhancement, it may be that IFN-α enhanced nonspecific immunity without significantly enhancing specific immunity. The ADV+IFN-α+GM-CSF group not only improved the level of IFN-γ secreted by HBSAG-specificCD4⁻Tcells, but also increased the level of CD8⁺ T secretion of HBSAG-specific cells. However, for the ADV+IFN-α+GM-CSF+vaccine group, in addition to HBV-specific IFN-γ⁺CD4⁺T cells and IFN-γ⁺ CD8⁺T cells increased, but also produced anti-HBsAg. From the analysis of the correlation between Treg and IFN-γ⁺CD4⁺T cells and IFN-γ⁺ CD8⁺T cells, the ADV+IFN-α+GM-CSF group and the ADV+IFN-α+GM-CSF+vaccine group may activate the HBsAg specific cell immunity, which is the key to breaking the immune tolerance and to clearing the HBsAg. In addition, both the ADV+IFN-α+GM-CSF group and the ADV+IFN-α+GM-CSF+vaccine group were able to significantly promote the TH1/Treg and Th2/Treg ratios, while the ADV+IFN-α+GM-CSF group was higher than Th1/Th2 in promoting the ADV+IFN-α+GM-CSF+vaccine group, but the difference was not statistically significant, the results showed that 3×GM-CSF and 3×GM-CSF+vaccine had the advantage of inducing HBV specific cellular and humoral immunity. However, the clinical trials may still need to be expanded to verify the effectiveness of the therapy.

In conclusion, the design of HBV-specific T lymphocytes in vitro “10-day Enrichment method” can be from the cellular immunology point of view for the clinical treatment of CHB patients to evaluate, and can be used as a concomitant diagnostic method in order to fill the blank of routine biochemical and viral evaluation.

### Example 3

### Monitor Long-Term Stability of the Cell Immunoassay Kit

Materials and reagents in reference example 1, PBMC was derived from patients with chronic hepatitis B in YIC group. The detection criteria was mainly IFN-γ expression in a CD8⁺ T cell.

The stability of a cell immunoassay kit stored at 1 month, 3 months, 6 months, and 12 months was monitored. The 96-well pre-coated cell stimulation plate in the kit was stored at −20° C. and the other reagents were stored at 4° C.

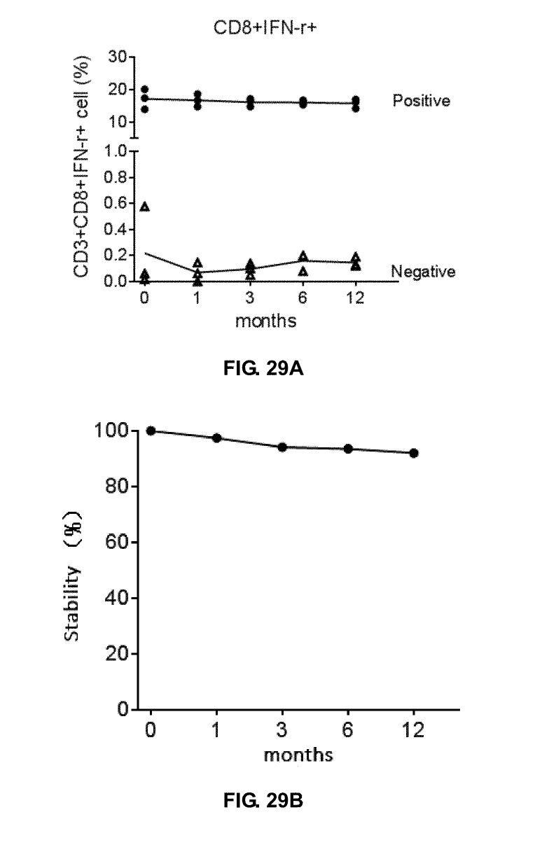

As shown in FIG. 29A, compared with day 0, the kits stored for 1 month, 3 months, 6 months, and 12 months were tested and the IFN-γ expression in CD8⁺ T cells in the positive stimulator wells did not change substantially, a negative stimulation well served as a control. Assuming that the stability of the kit was 100% on day 0, the stability of the kits preserved for 1 month, 3 months, 6 months, and 12 months remained above 90%. These results show that the cell immunoassay kit still had a good stability after one year of storage, as shown in FIG. 29B.

In the context above of embodiments of the present invention, CD8 S refers to the simultaneous use of two or three groups of T cell HBsAg epitope peptides.

The embodiments have been described in detail above, but these are only examples, and the present invention is not limited to the above-described embodiments. Any equivalent modifications and substitutions to the present invention are also within the scope of the present invention. Therefore, all equivalent changes and modifications without departing from the spirit and scope of the present invention should be covered by the scope of the present invention.

### Example 4

The Hepatitis B Virus (HBV) infection, which is detrimental to human health, is a significant infectious disease. In clinical discussion, terminating the replication of HBV continuously and the serotype transformation of the Hepatitis B e antigen (HBeAg), by the administration of PEG IFN-α or novel nucleoside analogues, may be considered as an acceptable recovery from the HBV infection. An undetectable Hepatitis B surface antigen (HBsAg) with or without the serotype transformation of HBsAg may be considered an ideal end point of clinical treatment. Therefore, efforts may be done to achieve the ideal cure end point among HBeAg serotype transformed patients. The natural rate of the HBeAg serotype transformation is 1-2%. Clinical research has shown that after five years of treatment, a Tenofovir HBsAg serotype was transformed in 23 of 266 HBeAg positive CHB patients. Further, research in China has shown that after treatment of Tenofovir combined with PEG IFN-α, the rate of HBsAg serotype transformation is 2.83% among HBeAg positive CHB patients. In another study, HBeAg negative CHB patients were administrated with a one year PEG IFN-αtreatment, and three years later HBsAg had disappeared in 8.7% of the patients with the HBsAg serotype transformation rate as 2.9%. The clinical data demonstrated that to eradicating HBsAg from patients is a tough challenge and novel drugs or immunotherapy are urgently needed for the cure of the HBV infection. Development of new strategies and novel drugs to reduce the HBsAg in CHB patients has been set as the main aim of the 12^{th} Five Year National Science and Technology Major Project in Infectious Disease.

In order to eliminate HBsAg a novel immunotherapy strategy was designed. In one embodiment of the strategy, one dose of GM-CSF may be injected every day for three consecutive days, and on the 4^{th} day, one dose of an HBV vaccine may be injected in the same injection site. This novel immunotherapy strategy may break the immune tolerance in HBV transgenic mice (Tg (A1b-1HBV) 44Bri/J). Both specific humoral and cellular immune were induced with the immunotherapy and 90% of the HBsAg was cleared in a plurality of transgenic mice. This immunotherapy strategy has been registered in Chinese Clinical Trial Registry (Registered code: ChiCTR-TRC-13003254; Registered name: Increase HBsAg Clearance Rate in HBeAg Positive Chronic Hepatitis B Patients with New Antiviral/Immunomoduratory Therapy; Web site: http://www.chictr.org.cn/showproj.aspx?proj=6305). The clinical experiment was supervised by the ethics committee of Huashan Hospital, Fudan University. All enrolled patients were signed with informed consent form.

Detection of two indicators, virology detection, which may include HBV-DNA detection and HBVM detection, and serology biochemical detection, which may include ALT detection, AST detection, γ-GT detection, albumin detection, and bilirubin detection, may be used to evaluate the curative effect of a clinical treatment of an HBV infection. Cellular immunity plays an important role during the treatment of an HBV infection, and there is still no well-established evaluation system which may be used to monitor the process of clinical treatment. In order to monitor the kinetics of the cellular immunity of the CHB patients which were enrolled in the clinical trial (ChiCTR-TRC-13003254) a systematic platform for the management of clinical bio-samples was built. A method, which named a 10-day enrichment, to collection PBMC from the patients was also developed. This system has the potential to stop the gap of clinical immune evaluation of an HBV infection treatment.

2. Materials and Methods

2.1 Clinical Experiment Design and Enrollment of CHB Patients.

Based on the principle of GCP, the clinical experiment was designed as multi-centered, randomized, prospective, and controlled. The drugs used in the experiment include Nucleoside Analogues (NA), Adefovir Dipivoxil (Adv), Interferon-α-2b (IFN-α), Granulocyte-Macrophage Colony-Stimulating Factor (GM-CSF) and HBV vaccine (VACCINE). The clinical experiment was registered in Chinese Clinical Trial Registry (Registered code: ChiCTR-TRC-13003254; Registered name: Increase HBsAg Clearance Rate in HBeAg Positive Chronic Hepatitis B Patients with New Antiviral/Immunomoduratory Therapy; Website: http://www.chictr.org.cn/showproj.aspx?proj=6305). The clinical experiment was supervised by the ethics committee of Huashan Hospital, Fudan University. All enrolled patients signed an informed consent form.

The experiment was conducted in Huashan Hospital and 104 CHB patients were enrolled. The patients already received clinical treatment before enrollment. Further, patients were randomly divided into 4 groups and the treatment lasted for 48 weeks. Patients were seen for a follow-up visit twice on weeks 60 and 72. All the patients must be treated with NA for at least 71 weeks.

Names of the divided group: (1) Treatment with adefovir (Adv); (2) Adv +IFN-α; (3) Adv+IFN-α+GM-CSF; (4) Adv+IFN-α+GM-CSF +VACCINE.

2.2 Inclusion and Exclusion Criteria

Inclusion criteria: Aged 18 to 65 years old, males or females; CHB patients whose HBeAg serotype transformed to negative, HBV-DNA cannot be detected, ALT returned to normal level after treatment with NA. The patients must be also given consolidation therapy for at least one year. The NA used for the treatment may include, singly or arbitrary combination, adefovir, Adv.

Exclusion criteria: Patients infected with Hepatitis A virus, Hepatitis C virus, Hepatitis D virus, Hepatitis E virus, and HIV; Patients having an allergic reaction to NA, Interferon, GM-CSF, and/or HBV Vaccine; Patients with cirrhosis or an Alpha fetoprotein level over 100 ng/ml; Patients with other liver related diseases, which may include autoimmune liver disease, alcoholic liver disease, non-alcoholic fatty liver disease, drug-induced hepatitis, and so on; Pregnant or lactating women; Patients with serum creatinine above the normal level, hemoglobin under 100 g/L, platelet count under 80×10⁹/L, neutrophil count under 1.5×10⁹/L; Patients with diseases in the heart, brain, kidney, retina, and/or muscle; Patients with endocrine system disease, autoimmune disease, and/or malignancy.

2.3 Randomization

Qualified experimental subjects were divided randomly into four groups. A random number was generated with a computer and combined with the treated drugs randomly. The candidates were randomly given an envelope which had a random number printed outside and the drug information printed inside. Investigators treated the candidate by the indication of drug information inside the envelope. Subjective selection or changing drugs was forbidden.

2.4 Drugs Information

TABLE 10
Drugs Information

| Drug Name | Manufacture | Size | |
|---|---|---|---|
| Adefovir Dipivoxil | | | |
| IFN-α | Kawin Technology | 5 | mIU/Dose |
| GM-CSF | Xiamen Amoytop | 75 | μg/Dose |
| | Biotech Co. LTD | | |
| HBV VACCINE | BioKangtai Co. LTD | 20 | μg/Dose |

2.5 Treatments

Group I: Oral administration with Adefovir once a day; Group II: in addition of the Adefovir treatments, add-on administrations of IFN-α by intramuscular injection once a day; Group III: in addition to the treatment of group II, add-on administrations of GM-CSF by subcutaneous injection for three continuous days; Group IV: in addition to the treatment of the group III, add-on administrations of HBV Vaccine one day after 3 days pretreatments of GM-CSF.

The administration of IFN-α, GM-CSF, and HBV Vaccine will last to week 48, and the Adv administration will last to week 72 with the follow-up visit. A scheme of the immune therapy is shown in FIG. 7.

2.6 Halt criteria: Poor compliance with the experiment subjects; Pregnancy of the experiment subjects; Adverse reaction of the experiment subjects; Quit of the experiment subjects; Death or cannot follow-up visited with the experiment subjects.

2.7 Evaluation of the Curative Effect and the Safety Assessment

2.7.1 The Main Indicator for the Evaluation of the Curative Effect

HBsAg transformed to negative; HBsAg serotype transformed (HBsAg<0.05 IU/mL and HBsAG>10 IU/mL); HBsAg decreased at least 10% of the basement.

2.7.2 Other Indicators for the Evaluation of the Curative Effect

HBV DNA cannot be detected or the level decreased at least 2 log 10 IU/mL compared with the basement on weeks 12, 24, 36, 48 and 72, the AST and ALT level at weeks 12, 24, 36, 48 and 72, and the histological response, which should with the inflammation and fibrosis rate under level 1, of the liver on week 48.

2.7.3 Indicators for Safety Assessment

Vital sign; Adverse reaction; Anaphylactic reaction; Clinical data; Physical examination.

4.2.8 Virology Detection and Biochemical Detection

Routine biochemistry (liver function, renal function); Blood cell analysis; Serum HBV DNA detection; and HBVM detection. All of the detection was conducted in the central laboratory of Huashan Hospital.

2.9 Clinical Cellular Immune Detection

Cellular detection was conducted in Key Laboratory of molecular virology, Ministry of education, Fudan University.

2.9.1 Reagents

TABLE 11
Reagents

| Name | Lot | Size | | Manufacture |
|---|---|---|---|---|
| Lymphocyte Separation | 1114546 | 599 | mL | Axis-shield |
| Medium | | | | |
| RPMI-1640 | 11875-093 | 500 | mL | Gibco |
| Fetal Bovine Serum | 10100-147 | 500 | mL | Gbico |
| Penicillin-Streptomycin | BL505A | 100 | mL | Wohong, |
| | | | | Nanjing |
| Anti-Human CD28 | 130-093-375 | 100 | μg/mL | Miltenyi |
| antibody | | | | Biotec |
| Recombinant human IL-2 | AE-200-02 | 100 | μg | Peprotech |
| Phorbol ester | P1585 | 1 | mg | Sigma |
| Lonomycin | 10634 | 2 | mg | Sigma |
| Brefeldin A | 555029 | 1 | mL | BD |
| PBS | SH30256.01 | 500 | mL | Hyclone |
| DMSO | 67-68-5 | 100 | mL | AppliChem |
| 4% PFA | G1101 | 500 | mL | Guge, Wuhan |
| Intracellular Staining | 421002 | 100 | mL | Biolegend |
| Permeabilization | | | | |
| Wash Buffer | | | | |
| FOXP3 Fix/Perm | 421401 | 30 | mL | Biolegend |
| Buffer (4x) | | | | |

2.9.2 Antibodies

TABLE 12
Antibodies

| | Clone | | Manufac- |
|---|---|---|---|
| Name | number | Isotype | turer |
| APC Anti-human CD4 | OKT4 | Mouse IgG2b, κ | eBioscience |
| FITC Anti-human CD4 | OKT4 | Mouse IgG2b, κ | eBioscience |
| APC Anti-human CD8 | SK1 | Mouse IgG1, κ | eBioscience |
| PE Anti-human IFN-γ | 4S.B3 | Mouse IgG1, κ | eBioscience |
| FITC Anti-human IL-2 | MQ1- | Rat IgG2a, κ | eBioscience |
| | 17H12 | | |
| APC Anti-human FOXP3 | PCH101 | Rat IgG2a, κ | eBioscience |
| PE Anti-human IL-10 | JES3-9D7 | Rat IgG1, κ | eBioscience |
| Brilliant Violet 421 ™ | MAb11 | Mouse IgG1, κ | BioLegend |
| Anti-human TNF-α | | | |
| Brilliant Violet 421 ™ | MP4-25D2 | Rat IgG1, κ | BioLegend |
| Anti-human IL-4 | | | |
| APC/Cy7 Anti-human | BL168 | Mouse IgG1, κ | BioLegend |
| IL-17A | | | |

2.9.3 Peptides

Peptides were synthetized in China Peptide Company LTD, with a purity of 95%. The sequence information was detailed in the Tables 2-4.

2.9.9 Blood Sampling, PBMC Isolation and T Cell In Vitro Enrichment

1) Blood sampling and transport

Intravenous blood samples were collected with an anticoagulant tube and shaken 5 to 10 times slowly. The samples may be kept erect and stored at room temperature. The transport may be finished within 2 hours.

2) Sample recording

Four Cryogenic Vials may be prepared, 3 for storage of plasma and 1 for storage of PBMC. Blood samples may be set for centrifuge with 1600 rpm, 10 min, at room temperature. Then plasma may be isolated and stored with 1 mL/vial at −80 ° C.

3) Isolation of PBMC

The rest of the sample was mixed with PBS to a volume of 20 mL and carefully transferred into a new tube with lymphocyte separation medium added before. Then the tube may be set for centrifuge with 2000 rpm, with both the accelerate rate and decelerate rate as zero, 30 min., and at room temperature.

4) Collection of PBMC

Abandon supernatant with 3 to 5 ml. Carefully collect the buffy coat cells and transfer into a new 15 mL tube. Sterile PBS may be added until 15 mL, set for centrifuge with 2000 rpm, with both the accelerate rate and decelerate rate as nine, 5 min., and at room temperature.

5) Preparation of 10% FBS-RPMI-1640

Eight mL of 10% FBS-RPMI-1640 may be prepared with 8 uL human IL-2 and 8 uL human CD28 added.

6) Detection of HLA-A2

PBMC may be stained with an anti-HLA-A2 antibody and detected by FACS.

7) Cell seeding and peptide stimulation

PBMC may be re-suspended with 10% FBS-RPMI-1640 and seeded into a 12-well plate with 1 mL/well. Specific peptides may be added at the same time with 1 μL per well. The plate may then be incubated in 5% CO2, 37° C.

2.9.5 Fluid Supplement

After five days incubation, 504, samples were taken from each well and stored at −80° C. Then 500 uL 10% FBS-RPMI-1640 containing 0.75 human IL-2 was added, and incubation was continued for another five days.

2.9.6 Isolation and Storage of the Enriched Cells

1) Preparation of 10% FBS-RPMI-1640 containing CD28 and BFA (Day 10). For one sample 4 mL 10% FBS-RPMI-1640 was added with 5 μL CD28 and 5 μL BFA.

2) Stimulation

After 10 days of enrichment, cells in a 12-well plate were transferred into a 1.5 mL EP tube and then centrifuged at 1500 rpm, 3 min. The supernatant was discarded and the cells were re-suspended with FBS-RPMI-1640 prepared in step 1. The suspended cells were then seeded into a 96-well plate, the plate being coated with PMA, N, CMV, S4, S8, and MIX, 90 uL/well with three repetitions. Then the plate was incubated in 5% CO2, 37° C. for 8 hours.

3) Storage

After 8 hours of incubation, the 96-well plate was set for centrifuge at 1500 rpm, 3 min. The supernatant was discarded and the cells were re-suspended with 200 μL cell freezing medium (10% DMSO with 90% FBS). Disassociation was performed by pipetting 3 to 5 times and sealing with parafilm. The cells were then set for gradient cryopreservation at 4° C. for 1 hour, −20° C. for 40 min and −80° C. thereafter.

2.9.7 Cell Staining and Detection

1) Cell recovery

The 96-well plate stored at −80° C. was incubated at 37° C. for about 20 to about 30 minutes. Then, the cells were centrifuged at 1600 rpm for 3 minutes. The supernatant was discarded and the cells were re-suspended with 150 μL PBS.

2) Transfer into another plate

In order to facilitate the staining, samples were transferred into another 96-well plate, which with was strictly marked before.

3) Cell surface marker staining

Panel 1, CD4-APC; Panel 2, CD8-APC; Panel 3, CD4-FITC. The dose of antibody was the recommended dose in the reagent manual. The plate was centrifuged at 1500 rpm for 3 minutes. The supernatant was discarded and the cells were re-suspended with staining buffer at 4° C. for 30 minutes.

4) Termination of the staining

200 μL PBS was added to terminate the staining and then was centrifuged at 1600 rpm for 3 minutes.

5) Cell fixation

Cells were set for fixation with 4% BFA for 10 min. For Treg staining, the cells were fixed with Foxp3 Transcription Factor Staining Buffer Kits. After fixation, the cells were centrifuged at 3000 rpm for 3 minutes. The supernatant was discarded and the cells were re-suspended with 200 μL PBS, at 3000 rpm for 3 minutes.

6) Intracellular staining

Panel 1/Panel 2, IFN-g-PE, TNF-a-BV421, IL-17-APC/cy7, IL-2-FITC; Panel 3, FOXP3-APC, IL-4-BV421, IL-10-PE. The dose of antibody was the recommended dose in the reagent manual. The cells were re-suspended with staining buffer at room temperature for 1.5 hours by shaking.

7) Termination of the intracellular staining

200 μL PBS was added in order to terminate the staining and then centrifuged at 1600 rpm for 3 minutes.

8) FACS detection

The detection was conducted with BD LSR Fortessa (BD Bioscience, USA). The voltage and compensation must be well adjusted. The result data was analyzed with FlowJo v7.6.3, and the results were shown as positive percentage.

2.10 Data Management and Statistical Analysis

2.10.1 Data Input

The data in our experiment were recorded with Case Report Form (CRF). The CRF can be fulfilled on the internet (URL: https://secure.eclinicalos.com/login?null). The data is open for all participated institutions. In order to avoid the disclosure of personal information, only the name and random number were used for registration.

2.10.2 Data Verification

After all the input of data was finished, a manual verification was then conducted. The administrator should give a report about research progress, check the inclusion and exclusion criteria, check the integrity, check the data consistency, and check the logicality of data and the adverse accident.

2.10.3 Statistical Analysis

Statistical analysis was conducted by SPSS v.19.0. The level of HBsAg was shown as logio. Cochran-Mantel-Haensel (CMH) was used for the comparison analysis among groups. If p<0.05 there was significant differences among the four groups, and then F-test was applied for analysis between two groups. Data in the same group, but with different time points, were analyzed by a paired t-test. Data with different groups were analyzed with ANOVO. A Pearson correlation analysis was used to compare the correlation between two different indicators. α=0.05 was used for a two-tailed significant difference test, and P≤0.05 meant that there was significant difference between two groups.

3. Results

3.1 Baseline Characteristics of Subjects

One hundred and four chronic hepatitis B patients were initially enrolled, and randomly assigned to 4 groups except for gender (the male ratio in the Adv group 1 was lower than in the other three groups), but the baseline data was not statistically significantly different for the four groups (p>0.05, Table 1). 10 patients, who failed to follow up in the study, had dropped off due to either withdrew content, or residence resettlements. 94 patients (90.4%, 94/104) completed the trial, 25 patients received in the ADV group, 24 in the ADV+IFN-α group, 22 in the ADV+IFN-α+GM-CSF group, and 23 in the ADV+IFN-α+GM-CSF+Vaccine group (where the vaccine is HBV vaccine), representing 26.6%, 25.5%, 23.4% and 24.5% of the total number of trials completed (see FIG. 8).

TABLE 13
Baseline data for disease characteristics of subjects

| | | | | | IFN-α + | IFN-α + ADV + | |
|---|---|---|---|---|---|---|---|
| | | | | IFN-α + | ADV + | GM-CSF + | |
| | | Patients | ADV | ADV | GM-CSF | VACCINE | |
| Characteristic | | (n = 94) | (n = 25) | (n = 24) | (n = 22) | (n = 23) | P value |
| Age | Median (SD) | 42.3(10.8) | 44.3(11.2) | 40.5(10.2) | 44.3(9.9) | 40.0(11.7) | 0.685 |
| Male | No. (%) | 82(84.5) | 18(72.0) | 24(100.0) | 20(90.9) | 20(87.0) | 0.042 |
| HbsAg (log₁₀ IU/mL) | Median (SD) | 2.52(0.88) | 2.83(0.76) | 2.38(1.08) | 2.56(0.73) | 2.26(0.83) | 0.178 |
| AST (U/L) | Median (SD) | 24.1(10.3) | 23.8(7.1) | 22.9(8.3) | 26.4(16.4) | 23.6(6.6) | 0.674 |
| ALT (U/L) | Median (SD) | 31.3(16.8) | 27.3(15.2) | 34.4(18.9) | 30.0(12.3) | 34.1(19.9) | 0.087 |
| WBC (×10⁹/L) | Median (SD) | 5.9(1.7) | 5.7(1.8) | 6.0(1.9) | 6.1(1.69) | 5.7(1.3) | 0.790 |
| HLA-A2 | Positive (%) | 50(53.2) | 14(56.0) | 13(54.2) | 11(50.0) | 12(52.2) | 0.992 |

3.2 Level of Serum HBsAg and ALT Kinetics in Subjects

In Group 1, no HBsAg decline from the baseline nor sero-conversion occurred, which was observed throughout the 48-week treatment and 72-week follow-up, and the ALT level did not change significantly neither.

In Groups 2 and 3, patients (3/24) showed a decline of HBsAg, accounting for 12.5% at the 24th week, but no more patients observed any HBsAg decline, and no more patients observed any HBsAg sero-conversion during the 48 weeks treatment and follow-up 72 weeks. There were changes of serum ALT with a decrease in the first weak, but peaked at the 24th week with a statistically significant difference when compared to the baseline (35.1±3.9 IU/L vs 56.8±8.2 IU/L, p<0.05), and further dropped to the baseline at the end of treatment at 48 weeks and at the 72 week follow-up.

In Group 3, 6 patients (6/22) showed a decline of HBsAg at the 24th week and throughout the end of the 72 week follow-up, which accounted for 27.3%. 1 out of these 6 patients showed HBsAg clearance (<0.07 IU/L) without sero-conversion. The level of serum ALT also showed a tendency to rise first and reached the highest peak at the 12th week with statistically significant difference from the baseline data (29.3±2.6 IU/L vs 47.2±6.3 IU/L, p<0.05). It declined at 48 weeks and reached the baseline level at 72 weeks.

In Group 4, 1 patient showed a decline of HBsAg, and another patient showed a decline of HBsAg with the sero-conversion (HBsAb at 692 IU/mL, with HBsAg for 7.2 IU/L) at the 24th week. At the end of the 48 week treatment, 2 more patients showed a decline of HBsAg, and 3 other patients had HBsAg sero-conversion (HBsAb at 90.0 IU/mL, 24.8 IU/mL and 433.9 IU/mL, respectively). At the end of follow-up, all of these 3 patients had HBsAg levels less than 10 IU/L, with HBsAb at 15.7 IU/mL, 15.7 IU/mL, and 222.7 IU/mL, respectively. The level of serum ALT fluctuated at an initially low level, and reached to the highest at the 24th week, but was not statistically significant when comparing with the baseline (33.8±4.1 IU/L vs 44.3±4.1 IU/L, p>0.05). The levels were returned to the baseline level at 48 weeks and at 72 weeks. (see FIGS. 9 and 10).

As shown in FIG. 9, zero weeks before immunotherapy was used as baseline. Serum HBsAg was detected by ELISA. Plot A represents HBsAg dynamics and each dot represent a patient. Plot B of FIG. 9 illustrates four groups with each patient treated with serum HBsAg changes. The color lines depict each group of HBsAg transformation or sero-conversion of the HBsAg in patients.

As shown in FIG. 10, zero weeks before immunotherapy was used as a baseline, with each dot representing a patient.

3.3 Clinical and Immunological Evaluation

Antigen specific cellular immunity may play an important role against chronic HBV infection and viral eradication. In order to understand the dynamic changes of cellular immunity during the treatment course, peripheral blood mononuclear cells (PBMC) samples were collected before the first treatment (i.e., at 0 weeks), weeks 4 (patients in the Group 1 were not collected), 12, 24, 36, and 48 during the treatments, and twice during the follow-up period at weeks 60 and 72. Isolated PBMCs at these time pointes were treated with human IL-2 and anti-human CD28 before stimulated with a pool of HBV-specific peptides for CD4 or CD8 T cells, CMV specific peptide as a positive control for 10-days in vitro culture with an additional human IL-2 on day 5 during the 10 day culture. After cells were collected and re-stimulated by a HBV-specific polypeptide for 8 hours, the levels of cytokines expressing CD4+and CD8+ T lymphocytes were intracellularly detected by flow cytometry. IL-2, IL-4, IL-10, IL-17a, FOXP3, IFN-γ, and TNF-α were analyzed in CD4+T cells, whereas IL-2, IL-17a, IFN-γ and TNF-α were analyzed in CD8+ T lymphocyte.

3.3.1 Quality Control of Clinical Immunology Test

In order to ensure the robustness of assays, positive controls for re-stimulation were employed, including PMA/Ionomycin, and human cytomegalovirus polypeptide (CMV, PP65). As shown in FIG. 4, cytokine releasing and expression of transcriptional factor FoxP3 were observed in both PMA/Ionomycin and CMV peptide stimulated cells.

The PBMC was re-stimulated by PMA/Ionomycin (100 ng/mL/1 μg/mL), or with a CMV specific peptide (1 μg/mL) for 8 hours, and Golgi blocker BFA (1:1000) was added in the last 4 hours. Intracellular staining was then performed and analyzed by flow cytometry.

3.3.2 Expression Level of Regulatory Factors in Treg Cells

Treatment of chronic hepatitis B is generally difficult due to systematic immune tolerance induced by HBV, and breaking such tolerance may be an important key to success. Regulatory T cell (Treg) plays an important role in maintaining the immune tolerance and FoxP3 may be the most important transcriptional factor in Treg. Therefore, FoxP3 expression dynamics in CD4+T lymphocytes was first analyzed in the four groups. As shown in FIG. 12, FoxP3 in CD4+T cell from 0 weeks to 72 weeks did not change significantly in the Adv group. In the Adv+IFN-α group, FoxP3 showed a first downward trend, then returned to baseline level after treatment at the 48th week. Both the Adv+IFN-α+GM-CSF group and the Adv+IFN-α+GM-CSF+Vaccine group were similar in that in the 24th week, FoxP3 decreased significantly (p<0.05). After treatment withdrawal, FoxP3 expression increased, but did not revive to the baseline level, and the difference was statistically significant (p<0.05).

IL-10 is key inhibitory cytokine secreted by Treg, and, therefore, IL-10 secretion dynamics in CD4+ T cell were analyzed and are shown in FIG. 13. In the Adv group, no significant change was observed. In the Adv+IFN-α group, IL-10 showed a trend of first ascending and then descending (p>0.05). Again, both the Adv+IFN-α+GM-CSF group and the Adv+IFN-α+GM-CSF+Vaccine group was similar that in that in the 24th week, IL-10 increased significantly (p<0.05), followed by a downward trend up to 72 weeks to the baseline level.

The x-axis of FIG. 12 is time points of 6 treatments (weeks 0, 4, 12, 24, 36 and, 48) and 2 follow-up points (weeks 60 and 72). The y-axis of FIG. 12 represents a percentage of FoxP3 positive CD4+T cells in total PBMC, each dot representing an individual patient. The green colors were labelled as the Adv group; Purple, the Adv+IFN-α group; Blue, the Adv+IFN-α+GM-CSF group; and orange, the Adv+IFN-α+GM-CSF+Vaccine group. Statistics were shown by the mean±standard error (MEAN±SEM), *(p<0.05) indicates statistical significance, and ns (p>0.05) indicates no significance.

The horizontal axis of FIG. 13 is time points of 6 treatments (weeks 0, 4, 12, 24, 36 and, 48) and 2 follow-up points (weeks 60 and 72). The y-axis of FIG. 13 represents a percentage of IL-10 positive CD4+T cells in total PBMC, each dot representing an individual patient. The green colors were labelled as the Adv group; Purple, the Adv+IFN-α group; Blue, the Adv+IFN-α+GM-CSF group; and orange, the Adv+IFN-α+GM-CSF+Vaccine group. Statistics were shown by the mean±standard error (MEAN±SEM), *(p<0.05) indicates statistical significance, and ns (p>0.05) indicates no significance.

3.3.3 Th1 Cytokines Stimulated by S-Peptide Pool in CD4⁺T Cells

Th1 cell immunization may play an important auxiliary role in chronic hepatitis B treatment, in addition to breaking immune tolerance. Therefore, next the PBMC after the stimulation of the S-polypeptide pool for cd4+t cells was analyzed. Three representative cytokines (i.e., Ifn-γ, IL-2, TNF-α) were analyzed and plotted in dynamics.

IFN-γ secretion in CD4+T cells in the Adv group showed no significant difference from 0 to 72 weeks. In the Adv+IFN-α group, IFN-γ showed a trend of ascending and descending from 0 weeks to 72 weeks (p>0.05). In both the Adv+IFN-α+GM-CSF group and the Adv+IFN-α+GM-CSF+Vaccine group, IFN-γ expression in the CD4+T cell increased significantly (p<0.05), and then decreased to the baseline level at 72 weeks, as shown in FIG. 14.

IL-2 and TNF-α showed no significant difference in the Adv group. In the Adv+IFN-αgroup, the Adv+IFN-α+GM-CSF group, and the Adv+IFN-α+GM-CSF+Vaccine group, there was a slight increase in the course of treatment without significance (p>0.05), and then gradually reverted to the baseline level, as shown in FIG. 14.

The x-axis of FIG. 14 is time points of 6 treatments (weeks 0, 4, 12, 24, 36 and, 48) and 2 follow-up points (weeks 60 and 72). The y-axis of FIG. 14 represents a percentage of IFN-γ, IL-2, or TNF-α positive CD4+T cells in total PBMC, each dot representing an individual patient. The green colors were labelled as the Adv group; Purple, the Adv+IFN-α group; Blue, the Adv+IFN-α+GM-CSF group; and orange, the Adv+IFN-α+GM-CSF+Vaccine group. Statistics were shown by the mean±standard error (MEAN±SEM), *(p<0.05) indicates statistical significance, and ns (p>0.05) indicates no significance.

3.3.4 Th2 Cytokines Stimulated by S-Peptide in CD4+T Cells

Th2 cells mainly function by helping B cells produce antibodies. In Result 4.3.2, it has been found that the Adv+IFN-α+GM-CSF+Vaccine group produced Anti-HBsAg antibodies, whereas the remaining three groups did not. Here, Th2 cells re-stimulated by the S-polypeptide pool with representative cytokine IL-4 dynamics was further analyzed. IL-4 showed no difference in the Adv group. In the Adv+IFN-α group, the Adv+IFN-α+GM-CSF group, and the Adv+IFN-α+GM-CSF+Vaccine group, IL-4 had a slight increase without significance (p>0.05), and then gradually reverted to the baseline level, as shown in FIG. 15.

The x-axis of FIG. 15 is time points of 6 treatments (weeks 0, 4, 12, 24, 36 and, 48) and 2 follow-up points (weeks 60 and 72). The y-axis of FIG. 15 represents a percentage of IL-4 positive CD4+T cells in total PBMC, each dot representing an individual patient. The green colors were labelled as the Adv group; Purple, the Adv+IFN-α group; Blue, the Adv+IFN-α+GM-CSF group; and orange, the Adv+IFN-α+GM-CSF+Vaccine group. Statistics were shown by the mean±standard error (MEAN±SEM), *(p<0.05) indicates statistical significance, and ns (p>0.05) indicates no significance.

3.3.5 Th17 Cytokines Stimulated by S-Peptide in CD4+T Cells

Next, the expression levels of TH17 cells in 0, 4, 12, 24, 36, 48, 60 and 72 weeks after the stimulation of the CD4+T cell by the PBMC peptide pool was studied in four treatment groups. There was no significant change in the IL-17A in the Adv group. Further, in the Adv+IFN-α group, the Adv+IFN-α+GM-CSF group, and the Adv+IFN-αGM-CSF+Vaccine group were slightly elevated in the course of treatment without significance (p>0.05) and was gradually dropped after 48 weeks of treatment, back to baseline level at 72 weeks, as shown in FIG. 16.

The x-axis of FIG. 16 is time points of 6 treatments (weeks 0, 4, 12, 24, 36 and, 48) and 2 follow-up points (weeks 60 and 72). The y-axis of FIG. 16 represents a percentage of IL-17 positive CD4+T cells in total PBMC, each dot representing an individual patient. The green colors were labelled as the Adv group; Purple, the Adv+IFN-α group; Blue, the Adv+IFN-α+GM-CSF group; and orange, the Adv+IFN-α+GM-CSF+Vaccine group. Statistics were shown by the mean±standard error (MEAN±SEM), *(p<0.05) indicates statistical significance, and ns (p>0.05) indicates no significance.

3.3.6 Tc1 Cytokines Stimulated by S-Peptide in CD8+T Cells

TC1 cell-biased immune response is a direct indication of HBV clearance. Three representative cytokines for TC1, IFN-γ, IL-2, and TNF-α and their dynamic changes, after the CD8+ T cells re-stimulated with a pool of HLA-A2⁺ or HLA-A2⁻ HBsAg peptides, was analyzed and plotted.

IFN-γ secreted by CD8+ T cells showed no difference in the Adv group. Further, the Adv+IFN-α group showed a trend of ascending and descending from 0 weeks to 72 weeks, without significance (p>0.05). In the Adv+IFN-α+GM-CSF group and the Adv+IFN-α+GM-CSF+Vaccine group, the expression of CD8+ T cell IFN-γ increased significantly (p<0.05) in 24 week and then decreased until 72 weeks to the baseline level, as shown in FIG. 17.

No significant difference was found in the IL-2 and TNF-α secreted by CD8+ T cells in the Adv group. In the Adv+IFN-α group, the Adv+IFN-α+GM-CSF group, and the Adv+IFN-α+GM-CSF+Vaccine group, both cytokines had a slightly higher high level in the course of treatment without significance (p>0.05), and then gradually reverted to the baseline level, as shown in FIG. 17.

The x-axis of FIG. 17 is time points of 6 treatments (weeks 0, 4, 12, 24, 36 and, 48) and 2 follow-up points (weeks 60 and 72). The y-axis of FIG. 17 represents a percentage of IFN-γ, IL-2, and TNF-α positive CD8+ T cells in total PBMC, each dot representing an individual patient. The green colors were labelled as the Adv group; Purple, the Adv+IFN-α group; Blue, the Adv+IFN-α+GM-CSF group; and orange, the Adv+IFN-α+GM-CSF+Vaccine group. Statistics were shown by the mean±standard error (MEAN±SEM), *(p<0.05) indicates statistical significance, and ns (p>0.05) indicates no significance.

3.3.7 TC17 Cytokines Stimulated by S-Peptide in CD8+T Cells

TC17 immune response represents an inflammatory response. Then, TC17 cells after the stimulation with either HLA-A2⁺ or HLA-A2⁻ HBsAg peptide pool was studied. Again, IL-17A showed no significance in the ADV group and the Adv+IFN-α groups. IL-17A levels were slightly elevated in the Adv+IFN-α+GM-CSF group and the Adv+IFN-α+GM-CSF+Vaccine group, but was not significant (p>0.05), and gradually reduced after 48 weeks of treatments and back to its baseline level at 72 weeks, as shown in FIG. 18.

The x-axis of FIG. 18 is time points of 6 treatments (weeks 0, 4, 12, 24, 36 and, 48) and 2 follow-up points (weeks 60 and 72). The y-axis of FIG. 18 represents a percentage of IL-17A positive CD8+ T cells in total PBMC, each dot representing an individual patient. The green colors were labelled as the Adv Group; Purple, the Adv+IFN-α group; Blue, the Adv+IFN-α+GM-CSF group; and orange, the Adv+IFN-α+GM-CSF+Vaccine group. Statistics were shown by the mean±standard error (MEAN±SEM), *(p<0.05) indicates statistical significance, and ns (p>0.05) indicates no significance.

3.3.8 Tc1 Cytokines in CD8+T Cells After Restimulation

The HBsAg protein vaccine was used in clinical trials to further understand the responsiveness of the core antigen (HBcAg) and polymerase (Pol) in an embodiment of the treatment, as described herein. Thus, CD8+T cells after stimulation of the HLA-A2+ (HBsAg+HBcAg+pol) mixed peptides were analyzed. IFN-γ (see plot A of FIG. 19), IL-2 (see plot B of FIG. 42), TNF-α (see plot C of FIG. 19) secreted by CD8+T cells showed no statistical significance (p>0.05).

The x-axis of FIG. 19 is time points of 6 treatments (weeks 0, 4, 12, 24, 36 and, 48) and 2 follow-up points (weeks 60 and 72). The y-axis of FIG. 19 represents a percentage of IFN-γ, IL-2, and TNF-α positive CD8+ T cells in total PBMC, each dot representing an individual patient. The green colors were labelled as the Adv Group; Purple, the Adv+IFN-α group; Blue, the Adv+IFN-α+GM-CSF group; and orange, the Adv+IFN-α+GM-CSF+Vaccine group. Statistics were shown by the mean±standard error (MEAN±SEM), *(p<0.05) indicates statistical significance, and ns (p>0.05) indicates no significance.

3.3.9 TC17 Cytokines in CD8+T Cells After (HBsAg+HBcAg+pol) Re-Stimulation.

The expression levels of Tc17 cells in 0, 4, 12, 24, 36, 48, 60 and 72 weeks after re-stimulation by HLA-A2+(HiBsAg+HBcAg+pol) mixed peptide were studied. For the IL-17a of the CD8+ T cells, there were no statistical significance (p>0.05), as shown in FIG. 20.

The x-axis of FIG. 20 is time points of 6 treatments (weeks 0, 4, 12, 24, 36 and 48) and 2 follow-up points (weeks 60 and 72). The y-axis of FIG. 20 represents a percentage of IL-17A positive CD8+T cells in total PBMC, each dot representing an individual patient. The green colors were labelled as the Adv Group; Purple, the Adv+IFN-α group; Blue, the Adv+IFN-α+GM-CSF group; and orange, the Adv+IFN-α+GM-CSF+Vaccine group. Statistics were shown by the mean±standard error (MEAN±SEM), *(p<0.05) indicates statistical significance, and ns (p>0.05) indicates no significance.

3.3.10 Dynamics and Correlation Between HBsAg and Treg

HBsAg Treg cell may be known to contribute to immune tolerance. In order to understand whether the level of HBsAg affects Treg level after treatment, the dynamic changes in the level of HBsAg and Treg in the course of treatments and follow-ups were analyzed. It was observed that both levels of HBsAg and Tregs did not decrease significantly throughout the treatments and follow-up in the Adv group, however, the level of HBsAg and Tregs decreased in a parallel manner during the treatments, but slightly raised in the follow-ups in the groups with Adv+IFN-α, Adv+IFN-α+GM-CSF, or Adv+IFN-α+GM-CSF+Vaccine (see FIG. 21).

The x-axis of FIG. 21 are time points of 6 treatments (weeks 0, 4, 12, 24, 36 and 48) and 2 follow-up points (weeks 60 and 72). The left y-axis of FIG. 21 are the HBsAg levels, and the right y-axes of FIG. 21 are Treg levels. The results were expressed by Mean±Sem. The red line is the HBsAg, and the black line is the Treg.

Having demonstrated the levels of HBsAg and FoxP3 were decreased concurrently during the treatments, and mostly notable at 24 weeks in the Adv+IFN-α group, the Adv+IFN-α+GM-CSF group, and the Adv+IFN-α+GM-CSF+Vaccine group, the Pearson correlation analysis was employed to confirm these correlations. As analysis results show in FIG. 22, both the levels of HBsAg and Tregs are positively correlated at 24 weeks in all three groups, but with various degrees. There was a positive, but modest correlation between HBsAg and Treg in the Adv group (r=0.413, p<0.05), also in the Adv+IFN-α group (r=0.466, p<0.05), in the Adv+IFN-α+GM-CSF Group (r=0.469, p<0.05), and as well in the Adv+IFN-α+GM-CSF+Vaccine group (r=0.437, p<0.05). As a result, the correlation between the HBsAg and Treg may indicate that HBsAg is an important factor that influences the immune tolerance.

3.3.11 Recipicol Correlation Between Percentage of FoxP3 and IFN-γ Expressing CD4 T Cells

As FoxP3 is the key marker for the inhibitory Tregs, while the inflammatory IFN-γ is a key anti-viral functional factor, the correlation between these two in HBsAg specific CD4+ T cells was analyzed. The dynamic changes in the level of the FoxP3+CD4+T cells were gradually reduced while the level of IFN-γ expressing CD4+T cells showed opposite changes during the treatments, as shown in FIG. 23. These reciprocal changes were only seen in the Adv+IFN-α+GM-CSF group and the Adv+IFN-α+GM-CSF+Vaccine group, but was not seen in the Adv group or the Adv+IFN-α group, even though there were a short periodic upswing at weeks 24 for the IFN-γ expressing in the Adv+IFN-α group. The IFN-γ+CD4 T cells reached a maximum level at weeks 24, when the 4^{th} treatment being given during the reciprocal changes, and gradually reduced near to its base level at 72 weeks as the end of follow-up, whereas, the Tregs remained at a lower level compared with its initial point for both the Adv+IFN-α+GM-CSF group and the Adv+IFN-α+GM-CSF+Vaccine group.

The horizontal axis of FIG. 23 was 6 time points of treatment (0 weeks, 4 weeks, 12 weeks, 24 weeks, 36 weeks, 48 weeks) and 2 follow-up points (60 weeks, 72 weeks). The left ordinate of FIG. 23 was the FOXP3 level in CD4+T cells, and the right ordinate of FIG. 23 was the INF-γ expression level in CD4+T cells. The results were expressed in Mean±sem. The red lines represent IFN-γ+CD4+T cells, and the black lines represent FoxP3+CD4+T cells.

In order to further understand the relationship between FoxP3+CD4+T cells and IFN-γ+CD4+T cells, a Pearson correlation at 24 weeks was analyzed and is shown in FIG. 24. In the Adv group, no correlation between the FoxP3 level and the IFN-γ level in CD4+T cells was observed (p>0.05). In the Adv+IFN-α group, FoxP3 levels were weakly and negative correlated with IFN-γ (r=-0.463, p<0.05), and the same was observed in the Adv+IFN-α+GM-CSF group (r=−0.504, p<0.05), as well as in the Adv+IFN-α+GM-CSF+Vaccine group (r=0.442, 0.05). The reciprocal correlation between the level of FoxP3+and IFN-γ+CD4 T cells may indicate that level of IFN-γ+CD4+T cells may be one of the important cells to break the immune tolerance.

The horizontal axis of FIG. 24 was FOXP3 of CD4+T cells at 24 weeks. The ordinate axis of FIG. 24 was IFN-γ level in 24 weeks. The correlation was calculated with a Pearson analysis.

3.3.12 Dynamic Correlation Between FoxP3+CD8+ T Cells and IFN-γ+CD8+ T Cells.

From previous results, FOXP3 in CD4+T cells showed an up-down trend, while IFNγ in CD8+ T cells showed an opposite down-up trend. As IFN-γ+CD8+T cells played a key role in the process of HBV removal, the dynamics and correlation between Foxp3 in CD8+ T cells and IFN-γ in CD8+ T cells was analyzed, as shown in FIG. 25. In the Adv group, FoxP3 did not show significant fluctuation, neither did IFN-γ secretion. In the Adv+IFN-α group, FoxP3 showed a down-up trend, and IFN-γ secretion showed an up-down trend, especially at 24 weeks. In the Adv+IFN-α+GM-CSF group and the Adv+IFN-α+GM-CSF+Vaccine group, FoxP3 first declined and then increased, and, in contrast, IFN-γ first increased then declined.

The horizontal axis of FIG. 25 was 6 time points of treatment (0 weeks, 4 weeks, 12 weeks, 24 weeks, 36 weeks, 48 weeks) and 2 follow-up points (60 weeks, 72 weeks). The left ordinate of FIG. 25 was the FOXP3 level in CD4+T cells, and the right ordinate of FIG. 25 was the IFN-γ expression level in CD8+ T cells. The results were expressed in Mean±sem. The red lines represent IFN-γ+CD8+ T cells, and the black lines represent FOXP3+CD4+T cells.

It was determined that the Foxp3+CD4+T cells had the most obvious decline at 24 weeks in the the Adv+IFN-α+GM-CSF group and in the Adv+IFN-α+GM-CSF+Vaccine group. Further, this was confirmed by analyzing a Pearson correlation at week 24. There was no correlation between FoxP3+CD8+ T cells and IFN-γ+CD8+ T cells in the Adv group and the Adv+IFN-α group (p>0.05), but there was a negatively correlation in the Adv+IFN-α+GM-CSF group (r=-0.537, p<0.05) and in the Adv+IFN-α+GM-CSF+Vaccine group (r=0.439, p<0.05). The correlation between FoxP3 levels in the above CD4+T cells and IFN-γ levels in CD8+T cells showed that the Adv+IFN-α+GM-CSF group and the Adv+IFN-α+GM-CSF+Vaccine group may promote the IFN-γ production in CD8+T cells, and in turn may contribute to breaking the immune tolerance, the final elimination of HBV.

The horizontal axis of FIG. 26 was FOXP3 of CD4+T cells at 24 weeks. The ordinate axis of FIG. 26 was IFN-γ level in CD8+ T cells in 24 weeks. The correlation was calculated with a Pearson analysis.

4. Discussion

Hepatitis B Therapeutic vaccines are considered a potential immunotherapy for chronic hepatitis B, however, development of hepatitis B therapeutic vaccines may be challenging, especially with regard to breaking the immune tolerance of hepatitis B, and stimulating the body against HBV specific cellular immune function may be limited. One of the possibilities is the lack of an appropriate adjuvant. Here, this clinical study has two main tasks: first, to explore the efficacy and feasibility of GM-CSF as adjuvant for Hepatitis B therapeutic vaccine in the treatment of patients with chronic hepatitis B, and the second is to provide clinical immunology experimental technical plan and accompanying diagnosis basis for the evaluation of HBV interventions.

Cure of hepatitis B may be rooted on HBsAg transformation and sero-convertion. The natural conversion rate is 1-2% annually. Even polyethylene glycol interferon alpha and nucleosides antiviral drugs combined treatment was administrated, conversion rate may be as low as 5% per year. According to data as discussed herein, after 48 weeks treatment, sero-conversion rate of HBsAg in the Adv+IFN-α+GM-CSF+Vaccine group was 15% (the data unpublished), which was 3 times more than any other therapies, and, thus, the 3×GM-CSF+HBV Vaccine protocol with the addition of standard anti-viral treatments had advantages over other current treatments in treating chronic hepatitis B patients.

Regulatory T cells (Regulatory T cell, Treg) are one of the important causes of immune tolerance in HBV infection. Shrivastava and other studies showed that, compared with HBsAg negative and healthy people, the frequency of FoxP3+regulated T cells increased significantly in hbsag-positive neonates, suggesting that the level of HBsAg was positively correlated with Treg level. The decline of Treg is considered an important indicator in order to break immune tolerance. As may be seen by the results described herein, after Adv+IFN-αtreatment, HBsAg decreased and the Treg frequency was also associated with the HBsAg, which was consistent with other experimental results. When compared with the Adv+IFN-α group, the Treg frequency of the Adv+IFN-α+GM-CSF group and the Adv+IFN-α+GM-CSF+Vaccine group was significantly lower than that in the baseline (p<0.05), which showed that the 3× GM-CSF+Vaccine may be more effective at breaking the immune tolerance state of CHB patients.

Specific immune activation may be an important mechanism for the removal of HBV, including the enhancement of helper T cell activation and the increase of CTL ability and humoral immunity. In particular experiments described herein, the specific cellular immunity of the organism was basically unchanged after treatment with nucleosides analogues. After the addition of IFN-α, the body specific cellular immunity did not have a significant enhancement. IFN-αmay have enhanced nonspecific immunity without significantly enhancing specific immunity. The Adv+IFN-α+GM-CSF group not only improved the level of IFN-γ secreted by HBsAg-specific CD4+T cells, but also increased the level of CD8+T secretion of HBSAG-specific cells. However, for the Adv+IFN-α+GM-CSF+Vaccine group, in addition to HBV-specific IFN-γ+CD4+T cells and IFN-γ+CD8+T cells increasing, an anti-hbsag was produced. From the analysis of the correlation between Treg and IFN-γ+CD4+T cells and IFN-γ+CD8+T cells, the Adv+IFN-α+GM-CSF Group and the Adv+ group, and the Adv+IFN-α+GM-CSF+Vaccine group may activate the HBsAg specific cell immunity, which is the key to breaking the immune tolerance and may clear the HBsAg. In addition, both the Adv+IFN-α+GM-CSF group and the Adv+IFN-α+GM-CSF+Vaccine group may be able to significantly promote the TH1/treg and th2/treg ratios, while the Adv+IFN-α+GM-CSF group may have higher th1/th2 in promoting the Adv+IFN-α+GM-CSF+Vaccine group, however, the difference was not statistically significant. Further, the results showed that 3× GM-CSF and 3× GM-CSF+Vaccine had the advantage of inducing HBV specific cellular and humoral immunity.

In conclusion, the design of HBV-specific T lymphocytes in vitro “10-day Enrichment method” may be from the cellular immunology point of view for the clinical treatment of CHB patients to evaluate, and can be used as a concomitant diagnostic method to fill the blank of routine biochemical and viral evaluation.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A cellular immunology assay kit for evaluating the efficacy of vaccines against a virus, the cellular immunology assay kit comprising a MHC restrictive viral antigen peptide.

2. The cellular immunology assay kit of claim 1, wherein the virus is pathogenic to humans and animals.

3. The cell immunological assay kit of claim 1, wherein the virus is the hepatitis B virus.

4. The cell immunological assay kit of claim 1, wherein the MHC restrictive viral antigen peptide is selected from the group consisting of a viral antigen CD4⁺ T cell epitope peptide, HBsAg, and a CD8⁺ T cell epitope HBsAg peptide.

5. The cell immunological assay kit of claim 1, wherein an HBsAg for a CD4⁺ T cell epitope peptide includes one or more of the following amino acid sequences: FFLLTRILTI (SEQ ID NO:1); FFLLTRILTIPQSLD (SEQ ID NO:2); TSLNFLGGTTVCLGQ (SEQ ID NO:3); QSPTSNHSPTSCPPIC (SEQ ID NO:4); CTTPAQGNSMFPSC (SEQ ID NO:5); CTKPTDGN (SEQ ID NO:33); WASVRFSW (SEQ ID NO:6); LLPIFFCLW (SEQ ID NO:7).

6. The cell immunological assay kit of claim 1, wherein an HBsAg for a CD8⁺ T cell epitope peptide includes one or more of the following amino acid sequences: VLQAGFFLL (SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); PFVQWFVGL (SEQ ID NO:32).

7. The cell immunological assay kit of claim 6, wherein any group or groups of the HBsAg CD8⁺T cells include one or more of the following amino acid sequence groups (A)-(C):
A) a CD8+T cell epitope peptides A2 HBsAg comprising one or more of the following amino acid sequences: VLQAGFFLL(SEQ ID NO:8); FLLTRILTI (SEQ ID NO:9); FLGGTPVCL (SEQ ID NO:10); LLCLIFLLV (SEQ ID NO:11); LVLLDYQGML (SEQ ID NO:12); LLDYQGMLPV (SEQ ID NO:13); WLSLLVPFV (SEQ ID NO:14); GLSPTVWLSV (SEQ ID NO:15); SIVSPFIPLL (SEQ ID NO:16); ILSPFLPLL (SEQ ID NO:17);
B) a CD8⁻T cell HBV mixed phenotypic peptide comprising one or more of the following amino acid sequences: FLLTRILTI (SEQ ID NO:9); WLSLLVPFV (SEQ ID NO:14); GLSPTVWLSV (SEQ ID NO:15); LLVPFVQWFV (SEQ ID NO:18); FLPSDFFPSI (SEQ ID NO:19); FLPSDFFPSV (SEQ ID NO:20); CLTFGRETV (SEQ ID NO:21); EYLVSFGVW (SEQ ID NO:22); TPPATRPPNAPIL (SEQ ID NO:23); KYTSFPWL (SEQ ID NO:24); and
C) a CD8⁺ T cell non-A2 HBsAg mixed epitope peptide comprising one or more of the following amino acid sequences: IPIPSSWAF (SEQ ID NO:25); WMMWYWGPSLY (SEQ ID NO:26); ILLLCLIFLL (SEQ ID NO:27); RWMCLRRFII (SEQ ID NO:28); RFSWLSLLVPF (SEQ ID NO:29); LYNILSPFL (SEQ ID NO:30); PFLPLLPIF (SEQ ID NO:31); PFVQWFVGL (SEQ ID NO:32).

8. The cell immunological assay kit of claim 1, wherein the cell immunological assay kit further comprises a test cell comprising a co-stimulatory signal.

9. The cell immunoassay assay kit of claim 8, wherein the co-stimulatory signals are anti-CD3 and anti-CD28 antibodies.

10. The cell immunological assay kit of claim 1, wherein the cell immunological assay kit further comprises a protein transport blocker.

11. The cell immunological assay kit of claim 1, wherein the cell immunological assay kit further comprises one or more pipetting apparatuses, a centrifuge tube, the cell culture vessel.

12. A method for storing a kit for cell immunology detection according to claim 1, wherein the method comprises the steps of pre-coating the kit with a cell stimulation plate stored at a temperature equal to or below −20° C., and storing the other reagents at a second temperature equal to or below 4° C.

13. A method of evaluating immunogenicity of a vaccine or immunotherapeutic composition by using the cell immunological assay kit of claims 1-11.

14. A method of evaluating immunogenicity of a vaccine or immunotherapeutic composition, the method comprising the steps of:
a) administering to a subject a vaccine or an immunotherapeutic composition;
b) obtaining from the subject a sample;
c) contacting the sample with a MHC restrictive viral antigen peptide compositions; and
d) evaluating immunogenicity of the subject.
